(19) United States
(12) Patent Application Publication
(10) Pub. No.: US 2026/0036588 A1
(43) Pub. Date: Feb. 5, 2026

(54) METHODS FOR LARGE TISSUE LABELING, CLEARING AND IMAGING USING ANTIBIODIES

(71) Applicant: Deep Piction GmbH, Munich (DE)

(72) Inventors: Ali Ertürk, Munich (DE); Hongcheng Mai, Munich (DE); Jie Luo, Munich (DE)

(21) Appl. No.: 19/115,109

(22) PCT Filed: Sep. 29, 2023
(86) PCT No.: PCT/EP2023/077157

§ 371 Date: Mar. 25, 2025

(30) Foreign Application Priority Data
Sep. 30, 2022 (EP) 22199276.1

(51) Int. Cl. G01N 33/58

(52) U.S. Cl. CPC G01N 33/582

(57) ABSTRACT

The present invention relates to methods for large tissue labeling, clearing and/or imaging using labeling agents such as antibodies, and uses and products related thereto. The present invention includes inter alia methods for preparing an animal tissue for fluorescence microscopy, an animal tissue obtainable by said methods, methods for analyzing said animal tissues, and methods for the detection of metastases, for analyzing the biodistribution of a biopharmaceutical drug, and for analyzing the biodistribution of nanoparticles. The methods for preparing an animal tissue according to the present invention encompass whole-body labeling, clearing and imaging methods. The methods of the invention are advantageous in that they, for instance, allow the visualization of single cells within mammalian tissues, including whole mouse bodies or other large tissues, tumor metastases at the single cell level and of the distribution of biopharmaceutical drugs (e.g. the distribution of cancer-targeting therapeutic antibodies in whole animals such as intact mice) at single cell level in whole mouse using labeling agents such as antibodies.

### FIELD OF THE INVENTION

The present invention relates to methods for large tissue labeling, clearing and/or imaging using labeling agents such as antibodies, and uses and products related thereto. The present invention includes inter alia methods for preparing an animal tissue for fluorescence microscopy, an animal tissue obtainable by said methods, methods for analyzing said animal tissues, and methods for the detection of metastases, for analyzing the biodistribution of a biopharmaceutical drug, and for analyzing the biodistribution of nanoparticles. The methods for preparing an animal tissue according to the present invention encompass whole-body labeling, clearing and imaging methods. The methods of the invention are advantageous in that they, for instance, allow the visualization of single cells within mammalian tissues, including whole mouse bodies or other large tissues, tumor metastases at the single cell level and of the distribution of biopharmaceutical drugs (e.g. the distribution of cancer-targeting therapeutic antibodies in whole animals such as intact mice) at single cell level in whole mouse using labeling agents such as antibodies.

### BACKGROUND

More than a century of dedicated work has provided a detailed understanding of the gross anatomy of the human body and the body of common model organisms and has produced detailed histological maps of many individual organs. However, it remains challenging for a given experimental condition to map the distribution, connectivity and molecular makeup of cell types across the whole body. For example, while the nervous system is connected to every part of the mammalian body, we do not have the cellular level maps of the nervous system to uncover the intricate relationships among organs and between organs and the central nervous system¹⁻³. In addition, most methods of imaging nerves or other cells in the context of whole bodies rely on transgenic animals^{4, 5}, which severely limits the flexibility of experimental design.

Generating new transgenic animals to map changes in the distribution of relevant proteins is usually prohibitively expensive and time consuming. However, such whole-body connectivity maps will be needed to understand the functional interdependence between organ systems and how a disease starting from one part of the body impact the rest such as during neurodegeneration or systemic inflammation.

Whole-body imaging could capture cellular insights and provide integrated biological knowledge in healthy rodents. However, while the mouse is the mouse commonly used animal model, we still lack basic information about its body, namely how diverse organ and tissue systems are organized in the whole mouse.

Recent clearing methods enabled antibody labeling and imaging of intact tissues⁶, mouse organs⁷ and bodies^{3, 8-14}, chunk of human organs¹⁵, and even human embryos¹⁶, but we still lack suitable, widely-applicable labeling methods for whole mouse bodies. Prior whole-body imaging methods, such as CUBIC, PACT and uDISCO, enabled whole-body imaging, but they relied on transgenic expression of fluorescent proteins in a subset of cells, such as mice expressing Thy-1 EGFP in neurons¹⁷. vDISCO methods uses small antibodies called nanobodies ( 1/10 of IgG size) for whole mouse body labeling. In contrast to the thousands of conventional unconjugated antibodies developed in the last decades, very few nanobodies work in a histological setting.

Although homogeneous labeling of whole bodies with small molecules (e.g., DNA-labeling dyes) or nanobodies can be achieved by cardiac pumping of solutions though the mouse vasculature⁵ (e.g., as described in WO 2018/224289 A1), this has proven difficult for standard IgG antibodies as 1) the antibodies are degraded and/or precipitate during perfusion, 2) cannot homogenously penetrate different tissue layers including muscles and bones, and 3) the cell membranes are not maximally permeabilized for antibodies to penetrate deep into all tissues with diverse properties.

Thus, further improved and more versatile methods for the preparation and analysis of tissues including whole animals and large mammalian brains are needed. Specifically, a whole-mouse indirect immunolabeling using primary and secondary conventional antibodies would be a particularly valuable method for many biological applications, including whole-body mapping of cells of interests.

### SUMMARY OF THE INVENTION

Here, a new technique is provided, which allows high-resolution 3D imaging of the peripheral nervous system (PNS), lymphatic system, and vascular system in the whole animal (e.g., mouse) body. This technique, which underlies the present invention, is termed wildDISCO (immunolabeling with wildtype mice and DISCO clearing), and is a chemical method enhancing the penetration of standard labeling agents, such as antibodies (preferably >100 kDa, e.g., ˜150 kDa size), into the whole animal body (e.g., ˜2 cm thick for a mouse body). The method allows to perform cholesterol extraction for permeabilization to ensure homogeneous penetration and staining across the tissues of, for instance, the whole mouse body including muscles, bones, the brain, and the spinal cord. Combining whole-body antibody labeling with DISCO-based tissue clearing allows to provide body-wide maps of cell-type and protein distribution with unprecedented ease and will help to advance our understanding of biological systems.

wildDISCO can reveal integrated neuronal, vascular, and lymphatic networks. By using the technique, it is possible to observe the PNS innervation in most organs, including the heart, lung, liver, kidney, stomach, and intestine. Moreover, the vagal nerves innervate the gastrointestinal tract may be visualized. By using the technique, also the lymphatic capillaries heterogeneously penetrated in the center of intestine villi and regional specific 3D villi lymphatics network may be presented. Surprisingly, it was found that lymph nodes were innervated by a population of PN with immunomodulatory potential. By using the technique, also the organ-specific vascular patterns and a network of transcortical capillaries as the main support for multiple bones may be imaged. Thus, mapping whole mouse body systems can provide a roadmap for diverse studies, including the neural circuits, immunomodulation, and angiogenesis in the entire mammalian body.

The present invention further allows unbiased imaging of transparent whole mouse bodies at cellular resolution provides a comprehensive view of biological systems (nerve or lymphatic systems) in health and disease. wildDISCO does not rely on the transgenic expression of fluorescent proteins, and permits the use of off-the-shelf IgG antibodies to homogenously and simultaneously staining structures in the whole mouse body.

The present invention provides a versatile method. The mouse head is a perfect example of the versatility of the method as it combines hard (skull) and soft tissues (brain). Using wildDISCO, it is possible to map the lymphatic vessels in and around the brain parenchyma in intact mouse heads.

In summary, the wildDISCO technology achieves a homogeneous and simultaneous antibody staining throughout large tissues such as the entire mouse bodies. Previously inaccessible 3D anatomical information becomes possible (e.g., aided by the VR visualization), allowing a more comprehensive understanding of the initiation, progression, and extent of pathologies at the whole organism level in mice.

The inventors reasoned that imaging optically transparent tissues including mice could be useful as a powerful preclinical approach, e.g. to detect fluorescently labeled cancer cells and/or therapeutic antibodies at cellular resolution in the intact body. Typically, fluorescent labeling of cancer cells in vitro or in vivo is achieved by endogenous expression of fluorescent proteins such as GFP, YFP and mCherry, which emit light in the visible spectrum. However, many tissues in the body also show high autofluorescence in this range (Tuchin, 2016; Zipfel et al., 2003), which can hinder reliable detection of single cancer cells through centimeters-thick intact mouse body.

According to preferred embodiments of the invention, labeling cells such as cancer cells using antibodies that are tagged with fluorescent dyes with emission peaks particularly in the far-red range is advantageous in order to overcome such autofluorescence signals by providing higher signal-to-background ratios for reliable detection of single cells.

Towards this goal, the inventors developed an improved method for preparing an animal tissue for fluorescence microscopy. Preferably, the method uses whole-body labeling (e.g. immunolabeling) technology based on antibodies to specifically label endogenous cellular proteins with fluorochromes such as Alexa and Atto dyes, preferably in the far-red spectrum, without the need to rely on endogenously expressed fluorescent proteins. Organic solvent-based clearing methods such as whole-body DISCO clearing methods (see Pan et al., 2016, which is incorporated by reference in its entirety for all purposes) can be included in the methods of the invention. The methods of the invention are advantageous in that they allow to visualize cells such as cancer cells in intact see-through mice even in tissues with high autofluorescence.

The methods of the invention can, for instance, be used to assess tumor metastasis and the biodistribution of a cancer cell-targeting antibody, e.g. in mice. This finding can be exemplified, for instance, by using mice transplanted with human mammary carcinoma cells and injected with the therapeutic monoclonal antibody 6A10 directed against carbonic anhydrase XII (CA12) (see Battke et al., 2011; and Gondi et al., 2013, for a reference to this antibody, which are incorporated by reference in their entirety for all purposes). As such, the present invention allows to advantageously detect spontaneous metastases and monitor tumor drug-target interactions at the single-cell level in intact mice and further phenotyping of defined tumor microenvironments via rehydration of cleared tissues and subsequent antibody labeling.

The methods that can be used, for instance, for the analysis of micrometastases and therapeutic anti-tumor antibody distribution in tissues such as whole mouse bodies at cellular resolution. The methods of the invention are unbiased, because they allow to label and detect target molecules in animal tissues (such as, for instance, whole mice) at single-cell resolution without dissection of the animal tissue prior to analyzing the animal tissue. Advantageously, the animal tissues that can be prepared and analyzed at single-cell resolution according to the invention without prior dissection are larger than in previously known methods. Thus, biases introduced by the dissection of the tissue (and by a subsequent separate analysis of the different dissected parts of said animal tissues) are minimized by the methods of the present invention. For example, a bias that may be introduced by analyzing only selected organs, or parts of such organs, can be minimized by the methods of the present invention. In non-limiting embodiments, the organic solvent used by the methods according to the invention contributes to this advantageous effect, because allows to shrink the animal tissue to a smaller size and makes the animal tissue more accessible to fluorescence microscopy with microscope objectives at their given maximum working distance.

The methods of the invention are also advantageous compared to previous methods in that they allow to clear tissues including skin, e.g. whole adult mice including skin.

The methods of the invention are also advantageous in that they can readily be applied in diverse labs without the need for highly specialized equipment, since imaging even with commonly used epifluorescence microscopes enables detection of greater detail in intact see-through mice than can be visualized through bioluminescence imaging.

The methods of the invention can, for instance, also reduce the time and cost needed for investigation of tumor micrometastases at the cellular level in whole mouse bodies. In addition, because researchers can readily evaluate a whole mouse body instead of selected tissues/organs, and because of the high sensitivity of the methods (being able to identify and quantify single cells throughout the body) the number of mice used in research can also be reduced significantly with the methods of the invention. Thus, the methods of the invention presented here can foster the translation of new therapies into the clinic much more efficiently than traditional methods.

Furthermore, unlike known tissue clearing methods such as CUBIC and PACT methods which make the tissue fragile, the method for preparing an animal tissue for fluorescence microscopy according to the invention renders the animal tissue hard. Thus, advantageously, the animal tissue obtainable by the methods of the invention is suitable for dissection into different parts and further analysis of the parts after dissection by fluorescence microscopy. It will be understood that according to the invention, dissection of the animal tissue that is obtainable by the methods of the invention is oftentimes not needed, because the animal tissues that can be prepared and analyzed at single-cell resolution according to the invention without prior dissection are larger than in previously known methods. However, if dissection is desired, the animal tissue that is obtainable by the methods of the invention can advantageously be used. This would be particularly useful to further characterize micrometastases which have been identified by the methods of the invention, and their microenvironments after isolation.

### Tissue Labeling Such as Whole-Body Immunostaining Using Antibodies

Imaging endogenous proteins such as endogenous fluorescent proteins in thick biological tissues presents major challenges, including the autofluorescence in the blue-green spectra and bleaching during lengthy imaging and storage. In exemplary embodiments of the invention, to achieve high signal quality (e.g., for single tumor cell detection in the whole adult mice), it is possible to label a primary antibody (bound to endogenous proteins, e.g. endogenous proteins of cancer cells) with a secondary antibody as labeling agent, such as secondary antibody conjugated to a fluorochrome, e.g., an Atto or Alexa dye. This approach is advantageous in that it increases the signal-to-background ratio and allows the visualization of single cells in tissues, in particular even in centimeters-thick mouse bodies. According to the invention, it will be understood that the use of fluorochromes even further in the far-red or longer wavelength spectrum, such as near-infrared fluorochromes, can be used to further increase the imaging quality and potentially allow studying sub-cellular structures/molecules in whole mouse bodies (see Hong et al., 2017, which is incorporated by reference in its entirety for all purposes, for examples of suitable fluorochromes).

The invention uses labeling with fluorochrome-containing labeling agents (e.g. antibodies conjugated to a fluorochrome) that preferably have a molecular weight of more than 100 kDa, e.g., equal to or more than 110 kDa, equal to or more than 120 kDa, equal to or more than 130 kDa, or equal to or more than 140 kDa.

Antibodies which can be conjugated to a fluorochrome and used in the invention include, without limitation, IgG molecules (e.g., IgG1, IgG2, IgG3 or IgG4), IgD molecules, IgE molecules, IgA molecules and IgM molecules.

The term “antibody” as used herein refers to any functional antibody that is capable of specific binding to the antigen of interest, as generally outlined in chapter 7 of Paul, W. E. (Ed.).: Fundamental Immunology 2nd Ed. Raven Press, Ltd., New York 1989, which is incorporated herein by reference. Without particular limitation, the term “antibody” encompasses antibodies from any appropriate source species, including chicken and mammalian such as mouse, goat, non-human primate and human. The antibody can be a monoclonal or polyclonal antibody. Such antibodies can be prepared by methods well-known in the art. The term “antibody” also encompasses-without particular limitations-isolated antibodies and modified antibodies such as genetically engineered antibodies, e.g. chimeric humanized or human antibodies.

In one embodiment, new antibodies can be generated for the methods and uses of the invention to study pathologies that are affecting the whole body. For example, a labeling agent (e.g. an antibody), which could be used as an inflammation or infection marker, would help to collect unbiased readouts in whole mice for inflammatory disorders, such as multiple sclerosis or rheumatoid arthritis, or infectious diseases, affecting the entire body.

### Detection of Micrometastases According to the Invention

Unbiased high-throughput mapping of tumor micrometastases at cellular resolution, e.g. in entire rodent bodies, can be a valuable tool to uncover the biology behind the dissemination of tumor cells. In exemplary embodiments, the invention encompasses the wildDISCO method which can be used for volumetric imaging of tumor micrometastases in the entire mouse body. While the usage of a single plane laser-scanning light-sheet microscope is the most preferred embodiment of the method for analyzing according to the invention, e.g. to detect cancer cells in see-through mice, utilization of even standard fluorescence microscopes can also provide novel insights. In addition, epifluorescence imaging helps to perform a straightforward scan of cleared mouse bodies within minutes to determine regions of interests before collecting large datasets with light-sheet microscopes. Subsequent light-sheet microscopy imaging could focus only on organs/regions of interest based on epifluorescence data. This approach would significantly speed up the conducted studies and reduce the amount of data to be analyzed.

Advantageously, the methods of the invention can be suitable for detecting and mapping cancer metastases in whole mouse bodies at the cellular level, allowing identification of the precise locations of single disseminated cancer cells. The methods of the invention allow re-probing of identified metastatic tissue with conventional antibodies, gene-expression profiling via e.g. RNAseq and proteomics (by mass spectrometry).

Thus, according to the invention, the methods for preparing an animal tissue for fluorescence microscopy of the invention are advantageous in that they preserve proteins (functional epitopes) and DNA/RNA.

Therefore, the methods of the invention can enable further characterization and molecular screening of micrometastases and single tumor cells identified in distant organs. According to the invention, usage of molecular markers for specific subtypes of tumor cells such as cancer stem cells, or of inflammatory cells and extracellular matrix components from the tumor microenvironment, such as cancer associated fibroblasts, T cells and macrophages will help to determine their exact spatiotemporal distributions in tissues, e.g. whole rodent bodies, during metastasis.

### Analysis of the Biodistribution of Biopharmaceutical Drugs According to the Invention

While precise assessment of biopharmaceutical drug (e.g. antibody drug) biodistribution is critical for evaluating its specificity and utility for treatments such as tumor treatments, there have been no methods that can provide such information at the cellular level in the intact organism. Methods of the invention (which are in exemplary embodiments also referred to as the “wildDISCO” methods) as a novel tool that can be used to study not only the distribution of single tumor cells, but also of antibody based therapeutics. Methods of the invention can allow identification of antibody-targeted tumor cells, in particular in metastases in different organs, including lungs, kidney, brain and liver. The methods for analyzing of the invention can also be advantageous in that they can also be used to detect binding of biopharmaceutical drugs (e.g. therapeutic antibodies) to non-target tissues (such as non-cancerous tissues in the case of cancer therapeutic antibodies) to indicate potential off-target effects.

### Analysis of the Biodistribution of Nanoparticles According to the Invention

In exemplary method for analyzing the biodistribution of nanoparticles, Nanoparticles (DNA origami or carbon nanotubes) can be conjugated to polymers such as PEG to increase the circulation time and stability. They are may also be tagged by moieties such as antibodies, peptides, aptamers for targeting. For example, CpG peptides can be used to target them to immune cells. Finally, they can also be conjugated to fluorochromes (e.g., Alexa or Atto dyes) for use in accordance with the methods of the present invention. The conjugated nanoparticles can be dissolved in PBS at 200 nM-2 μM concentrations. Then 100-200 μL of this solution is injected to mice either i.v. or i.p. Subsequently the mice are perfused as early as 3 hours (or longer). The biodistribution of nanoparticles is assessed by the methods of the invention.

Thus, the invention provides an advantageous labeling and analysis platform. This platform, for instance, allows visualization and analysis of tumor micrometastases and antibody based therapies at single cell resolution in whole mouse bodies. Because the methods of the invention are time and cost efficient, they can be used to investigate various biomedical questions, e.g. biomedical questions related to various pathologies or developmental processes that affect the organism as a whole.

Accordingly, the present invention encompasses the following preferred embodiments:

### Embodiments

1. A method for preparing an animal tissue for fluorescence microscopy, the method comprising the following steps:
   a) Optionally decalcifying a fixed animal tissue with a solution for decalcification;
   b) Optionally decolorizing the fixed animal tissue with a solution for the removal of heme;
   c) Labeling a target molecule in the fixed animal tissue with a labeling solution comprising a fluorochrome-containing labeling agent capable of binding to said target molecule, said labeling agent preferably having a molecular weight of more than 100 kDa, to obtain a fixed animal tissue labeled with said fluorochrome-containing labeling agent,
      wherein the fixed animal tissue is treated with a permeabilization solution, preferably before and/or during said labeling of the target molecule in step c), and
      preferably wherein the permeabilization solution and the labeling solution are the same or different solutions,
         and
      wherein the permeabilization solution and/or the labeling solution comprises a cyclodextrin derivative; and
   d) optionally clearing the fixed animal tissue labeled with said fluorochrome-containing labeling agent with a clearing solution comprising an organic solvent; so as to obtain said animal tissue for fluorescence microscopy.
2. The method according to embodiment 1, wherein the cyclodextrin derivative has a structure represented by the following formula:

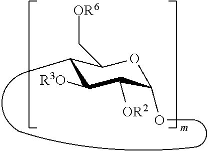

wherein:
m is 6 to 8;
R², R³ and R⁶ are each independently selected from H and optionally substituted alkyl; and
and the degree of substitution (DS), representing the average number of non-hydrogen groups R², R³ and R⁶ per glucopyranose unit, is 0 to 3.
3. The method according to embodiment 2, wherein the optionally substituted alkyl is a linear or branched C₁-C₆ alkyl optionally substituted by one or more groups selected from OH, SO₃H, SO₃Na, oxo and COOH.
4. The method according to any one of embodiments 2 and 3, wherein R², R³ and R⁶ are each independently selected from H and linear or branched C₁-C₄ alkyl optionally substituted by one or more groups selected from OH, SO₃H, SO₃Na, oxo and COOH.
5. The method according to any one of embodiments 2 to 4, wherein DS≥0.4.
6. The method according to any one of embodiments 2 to 5, wherein DS≥0.5.
7. The method according to any one of embodiments 2 to 6, wherein DS≥0.6.
8. The method according to any one of embodiments 2 to 7, wherein DS≥0.7.
9. The method according to any one of embodiments 2 to 8, wherein DS≥0.8.
10. The method according to any one of embodiments 2 to 9, wherein DS≥0.9.
11. The method according to any one of embodiments 2 to 10, wherein DS≥1.0.
12. The method according to any one of embodiments 2 to 11, wherein DS≥1.5.
13. The method according to any one of embodiments 2 to 12, wherein DS≥1.8.
14. The method according to any one of embodiments 2 to 13, wherein DS≥1.9.
15. The method according to any one of embodiments 2 to 14, wherein DS≥2.0.
16. The method according to any one of embodiments 2 to 15, wherein DS≥2.5.
17. The method according to any one of embodiments 2 to 16, wherein DS≤3.0.
18. The method according to any one of embodiments 2 to 17, wherein DS≤2.8.
19. The method according to any one of embodiments 2 to 18, wherein R², R³ and R⁶ are each independently selected from H and linear or branched C₁-C₆ alkyl optionally substituted by one or more oxo and/or OH.
20. The use according to any one the preceding embodiments, wherein the cyclodextrin derivative is not methyl-β-cyclodextrin with a substitution degree of 1.8.
21. The use according to any one the preceding embodiments, wherein the cyclodextrin derivative is not methyl-β-cyclodextrin with a substitution degree of <2.0.
22. The method according to any one of embodiments 2 to 21, with the proviso that when R², R³ and R⁶ are each selected from H and CH₃, then DS≥2.
23. The method according to any one of embodiments 2 to 22, wherein DS is 1.8 to 2.0.
24. The method according to any one of embodiments 2 to 23, wherein R², R³ and R⁶ are each independently selected from H and CH₃.
25. The method according to any one of embodiments 2 to 24, wherein the R², R³ and R⁶ are each independently selected from H and CH₃.
26. The method according to embodiment 25, wherein DS is 1.8.
27. The method according to any one of embodiments 2 to 23, wherein
   (a) R² and R⁶ are linear or branched C₁-C₆ alkyl, and R³ is H; or
   (b) R² and R³ are linear or branched C₁-C₆ alkyl, and R⁶ is H; or
   (c) R³ and R⁶ are linear or branched C₁-C₆ alkyl, and R² is H.
28. The method according to any one of embodiments 2 to 26, wherein R² and R⁶ are CH₃, and R³ is H.
29. The method according to any one of embodiments 2 to 26, wherein R², R³ and R⁶ are each independently selected from H and C(O)C₁₋₅ alkyl.
30. The method according to embodiment 29, wherein R², R³ and R⁶ are each independently selected from H and C(O)CH₃.
31. The method according to any one of embodiments 2 to 30, wherein DS is 2.5 to 3.0.
32. The method according to any one of embodiments 2 to 31, wherein R², R³ and R⁶ are each C(O)CH₃.
33. The method according to any one of embodiments 2 to 23, wherein R², R³ and R⁶ are each independently selected from H, —C₁₋₂alkyl(OH)C₁₋₃alkyl and —C₁₋₂alkyl-OH.
34. The method according to any one of embodiments 2 to 33, with the proviso that when the alkyl is substituted by one or more OH groups, then the alkyl is a linear or branched C₃-C₆ alkyl and/or DS≥0.9.
35. The method according to any one of embodiments 2 to 34, wherein DS is 0.8 to 3.0.
36. The method according to any one of embodiments 2 to 35, wherein DS is 0.9 to 3.0.
37. The method according to any one of embodiments 2 to 36, wherein R², R³ and R⁶ are each independently selected from H and —CH₂CH(OH)CH₃.
38. The method according to any one of embodiments 2 to 37, wherein DS is 0.9.
39. The method according to any one of embodiments 2 to 38, wherein R², R³ and R⁶ are each independently selected from H and —CH₂CH₂OH.
40. The method according to embodiment 39, wherein DS is 0.8.
41. The method according to any one of embodiments 2 to 40, wherein R², R³ and R⁶ are each independently selected from H and linear or branched C₁-C₅ alkyl optionally substituted by COOH.
42. The method according to any one of embodiments 2 to 41 wherein R², R³ and R⁶ are each independently selected from H and —C(O)—C₁₋₃alkyl-COOH.
43. The method according to any one of embodiments 2 to 42, wherein R², R³ and R⁶ are each independently selected from H and —C(O)CH₂CH₂COOH.
44. The method according to any one of embodiments 41 to 43, wherein DS is 0.4 to 0.7.
45. The method according to any one of embodiments 2 to 44, wherein DS is 0.5
46. The method according to any one of embodiments 2 to 45, wherein m is 6 or 8.
47. The method according to any one of embodiments 2 to 46, wherein m is 7.
48. The method according to any one of the preceding embodiments, wherein the cyclodextrin derivative is selected from (2-hydroxypropyl)-β-cyclodextrin, triacetyl-β-cyclodextrin, (2-hydroxyethyl)-β-cyclodextrin, heptakis (2,6-di-O-methyl)-β-cyclodextrin, succinyl-β-cyclodextrin γ-cyclodextrin and α-cyclodextrin; more preferably (2-hydroxypropyl)-β-cyclodextrin or heptakis (2,6-di-O-methyl)-β-cyclodextrin; most preferably heptakis (2,6-di-O-methyl)-β-cyclodextrin.
49. The method according to any one of the preceding embodiments, further comprising a blocking step for blocking unspecific antigen binding of antibodies, wherein the blocking step is performed by treating the fixed animal tissue with a blocking solution before the step of labeling.
50. The method according to embodiment 49, wherein the blocking solution comprises animal serum.
51. The method according to embodiment 50, wherein the animal serum is mammalian serum, preferably goat serum or donkey serum, more preferably goat serum.
52. The method according to any one of embodiments 49 to 51, wherein the blocking solution further comprises a surfactant.
53. The method according to embodiment 53, wherein the surfactant is a non-ionic surfactant, preferably Triton X-100 or IGEPAL CA-630, more preferably Triton X-100.
54. The method according to any one of embodiments 49 to 53, wherein the blocking solution comprises:
   the animal serum at a concentration of 1 to 15, preferably 3 to 10, more preferably 3% v/v; and/or
   the non-ionic surfactant at a concentration of 0.5 to 4, preferably 1 to 3, more preferably 2% w/v, in an aqueous buffer solution, preferably phosphate buffer saline (PBS), which optionally has buffer agent concentration of 0.05 to 0.2 M, preferably 0.08 to 1.2 M, more preferably 0.1 M.
55. The method according to anyone of the preceding embodiments, wherein the treating with permeabilization solution is performed simultaneously with the treating with blocking solution.
56. The method according to anyone of the preceding embodiments, wherein the permeabilization solution and the blocking solution are the same solution.
57. The method according to any one of the preceding embodiments, wherein the method comprises step a).
58. The method according to any one of the preceding embodiments, wherein in step a), the solution for decalcification is selected from a solution that comprises EDTA and NaHCO₃, a solution that comprises formic acid, a solution that comprises HNO₃, or a solution that comprises HCl.
59. The method according to any one of the preceding embodiments, wherein said fixed animal tissue is obtainable by fixation with a fixation solution comprising paraformaldehyde, optionally 4±2% w/v paraformaldehyde, and optionally heparin.
60. The method according to any one of embodiments 57 to 59, wherein a blocking step as defined in any one of embodiments 49 to 56 is performed after step a).
61. The method according to any one of the preceding embodiments, wherein the method comprises step b).
62. The method according to any one of the preceding embodiments, wherein step b) is performed by perfusing the fixed animal tissue with said solution for the removal of heme and/or wherein said solution for the removal of heme is a heme-chelating solution.
63. The method according to any one of the preceding embodiments, wherein in step b), said solution for the removal of heme comprises an aminoalcohol suitable for the removal of heme and optionally a surfactant.
64. The method according to embodiment 63, wherein said solution for the removal of heme comprises a surfactant, and wherein said surfactant is an ionic surfactant, a non-ionic surfactant, a zwitterionic surfactant, a chaotropic surfactant or a combination thereof.
65. The method according to embodiment 64, wherein said surfactant is an ionic surfactant which is sodium dodecyl sulfate or deoxycholate,
66. The method according to embodiment 64, wherein said surfactant is a non-ionic surfactant which is 4-(1,1,3,3-Tetramethylbutyl)phenyl-polyethylene glycol, t-Octylphenoxypolyethoxyethanol, Polyethylene glycol tert-octylphenyl ether or polyoxyethylene (20) sorbitan monolaurate.
67. The method according to embodiment 64, wherein said surfactant is a zwitterionic surfactant which is 3-[(3-Cholamidopropyl)dimethylammonio]-1-propanesulfonate hydrate.
68. The method according to embodiment 64, wherein said surfactant is a chaotropic surfactant which is urea.
69. The method according to any one of embodiments 63-68, wherein said aminoalcohol is N,N,N′,N′-tetrakis (2-hydroxypropyl)ethylenediamine, N-Butyldiethanolamine, N-Methyldiethanolamine, 4-(2-Hydroxyethyl) morpholine, N-Ethyldiethanolamine, 2-(Diisopropylamino) ethanol, 4-Methylmorpholine N-oxide or 1-(2-Hydroxyethyl) piperidine.
70. The method according to any one of embodiments 63-69, wherein said aminoalcohol is N,N,N′,N′-tetrakis (2-hydroxypropyl)ethylenediamine.
71. The method according to embodiment 70, wherein the solution for the removal of heme is a 1:2 or 1:3 dilution of the following reagent, preferably in 0.1 M PBS: 25 wt % urea, 25 wt % N,N,N′,N′-tetrakis (2-hydroxypropyl)ethylenediamine, 15 wt % Triton X-100 in 0.1 M PBS.
72. The method according to any one of the preceding embodiments, wherein in step b), said solution for the removal of heme comprises an oxidizing reagent for the oxidation of heme.
73. The method according to embodiment 72, wherein the oxidizing reagent for the oxidation of heme is benzyl peroxide, 3-chloroperoxybenzoic acid or magnesium monoperoxyphthalate hexahydrate.
74. The method according to embodiment 72, wherein the oxidizing reagent for the oxidation of heme is benzyl peroxide.
75. The method according to any of the preceding embodiments, wherein step b) is performed simultaneously with step c).
76. The method according to any of the preceding embodiments, wherein the solution for the removal of heme is the same solution as the permeabilization solution and/or the labeling solution.
77. The method according to any of the preceding embodiments, wherein the permeabilization solution and/or the labeling solution comprises a surfactant, and wherein said surfactant is an ionic surfactant, a non-ionic surfactant, a zwitterionic surfactant, a chaotropic surfactant or a combination thereof.
78. The method according to any of the preceding embodiments, wherein the permeabilization solution and/or the labeling solution comprises a non-ionic surfactant and/or a zwitterionic surfactant.
79. The method according to any of the preceding embodiments, wherein the permeabilization solution and/or the labeling solution comprises:
   non-ionic surfactant, preferably selected Triton X-100 and IGEPAL CA-630, more preferably Triton X-100; and
   optionally zwitterionic surfactant selected from CHAPS or CHAPSO, more preferably CHAPS.
80. The method according to any one of the preceding embodiments, wherein the fluorochrome-containing labeling agent has a molecular weight of equal to or more than 110 kDa.
81. The method according to any one of the preceding embodiments, wherein the fluorochrome-containing labeling agent has a molecular weight of equal to or more than 120 kDa.
82. The method according to any one of the preceding embodiments, wherein the fluorochrome-containing labeling agent has a molecular weight of equal to or more than 130 kDa.
83. The method according to any one of the preceding embodiments, wherein the fluorochrome-containing labeling agent has a molecular weight of equal to or more than 140 kDa.
84. The method according to any one of the preceding embodiments, wherein the fluorochrome-containing labeling agent has a molecular weight of equal to or more than 150 kDa.
85. The method according to any one of the preceding embodiments, wherein the fluorochrome-containing labeling agent has a molecular weight of 900 kDa or less.
86. The method according to any one of the preceding embodiments, wherein the fluorochrome-containing labeling agent has a molecular weight of 500 kDa or less.
87. The method according to any one of the preceding embodiments, wherein the fluorochrome-containing labeling agent has a molecular weight of 385 kDa or less.
88. The method according to any one of the preceding embodiments, wherein the fluorochrome-containing labeling agent has a molecular weight of 300 kDa or less.
89. The method according to any one of the preceding embodiments, wherein the fluorochrome-containing labeling agent has a molecular weight of 200 kDa or less.
90. The method according to any one of the preceding embodiments, wherein the fluorochrome-containing labeling agent has a molecular weight of 180 kDa or less.
91. The method according to any one of the preceding embodiments, wherein the fluorochrome-containing labeling agent has a molecular weight of 150 kDa or less.
92. The method according to any one of the preceding embodiments, wherein the fluorochrome-containing labeling agent has a molecular weight of 110 to 900 kDa.
93. The method according to any one of the preceding embodiments, wherein the fluorochrome-containing labeling agent has a molecular weight of 120 to 500 kDa.
94. The method according to any one of the preceding embodiments, wherein the fluorochrome-containing labeling agent has a molecular weight of 130 to 385 kDa.
95. The method according to any one of the preceding embodiments, wherein the fluorochrome-containing labeling agent has a molecular weight of 140 to 300 kDa.
96. The method according to any one of the preceding embodiments, wherein the fluorochrome-containing labeling agent has a molecular weight of 150 to 200 kDa.
97. The method according to any one of the preceding embodiments, wherein the fluorochrome-containing labeling agent has a molecular weight of 150 to 180 kDa.
98. The method according to any one of the preceding embodiments, wherein said fluorochrome is capable of emitting infrared or red fluorescence.
99. The method according to any one of the preceding embodiments, wherein said fluorochrome is capable of emitting near-infrared or far-red fluorescence.
100. The method according to any one of the preceding embodiments, wherein the emission maximum of said fluorochrome is at a wavelength of higher than 480 nm.
101. The method according to any one of embodiments 1 to 100, wherein the emission maximum of said fluorochrome is at a wavelength of higher than 500 nm.
102. The method according to any one of embodiments 1 to 101, wherein the emission maximum of said fluorochrome is at a wavelength of higher than 550 nm.
103. The method according to any one of embodiments 1 to 102, wherein the emission maximum of said fluorochrome is at a wavelength of higher than 590 nm.
104. The method according to any one of embodiments 1 to 103, wherein the emission maximum of said fluorochrome is at a wavelength of higher than 600 nm.
105. The method according to any one of embodiments 1 to 104, wherein the emission maximum of said fluorochrome is at a wavelength of higher than 640 nm.
106. The method according to any one of embodiments 1 to 105, wherein the emission maximum of said fluorochrome is at a wavelength of higher than 700 nm.
107. The method according to any one of embodiments 1 to 106, wherein the emission maximum of said fluorochrome is in a wavelength range of between 640 nm and 700 nm.
108. The method according to any one of embodiments 1 to 107, wherein the emission maximum of said fluorochrome is at a wavelength of lower than 1000 nm or lower than 900 nm.
109. The method according to any one of embodiments 1 to 108, wherein the emission maximum of said fluorochrome is at a wavelength of lower than 800 nm.
110. The method according to any one of the preceding embodiments, wherein the fluorochrome-containing labeling agent is an antibody conjugated to said fluorochrome, said antibody being capable of binding to said target molecule.
111. The method according to embodiment 110, wherein the antibody is an IgG, IgA, IgM, IgD or IgE.
111. The method according to embodiment 110 or 111, wherein the antibody is an IgG.
112. The method according to embodiment 111 wherein the antibody is an IgG1.
113. The method according to embodiment 111, wherein the antibody is an IgG2.
114. The method according to embodiment 111, wherein the antibody is an IgG3.
115. The method according to embodiment 111, wherein the antibody is an IgG4.
116. The method according to any one of the preceding embodiments, wherein step c) is performed by perfusing the fixed animal tissue with said labeling solution comprising the fluorochrome-containing labeling agent.
117. The method according to any one of the preceding embodiments, wherein the fluorochrome-containing labeling agent is fluorescent dyes, said fluorescent dyes being capable of binding to said target molecule.
118. The method according to any one of the preceding embodiments, wherein the fluorochome-containing labeling agent comprises a fluorescent dye, preferably wherein said fluorescent dye is Nissl, propidium iodide, methoxy-x04, Cresyl Violet acetate, Pyronin Y, Thiazin Red, lectin, Dil, Atto dyes, Alexa Fluor dyes, Cy dyes and To-pro3.
119. The method according to any one of the preceding embodiments, wherein the fluorochome-containing labeling agent comprises a fluorescent dye selected from Alexa Fluor 568, Alexa Fluor 647, Alexa Fluor 750, Atto 550, Atto 647, Cy7, Cy5 and Cy3.
120. The method according to any one of the preceding embodiments, wherein said organic solvent has a refractive index which deviates from the refractive index of the tissue of said animal by not more than 5%.
121. The method according to any one of the preceding embodiments, wherein said clearing solution comprising the organic solvent has a refractive index which deviates from the refractive index of the tissue of said animal by not more than 2%.
122. The method according to any one of the preceding embodiments, wherein said clearing solution comprising the organic solvent has a refractive index of between 1.500 and 1.600.
123. The method according to any one of the preceding embodiments, wherein said clearing solution comprising the organic solvent has a refractive index of between 1.520 and 1.580.
124. The method according to any one of the preceding embodiments, wherein said organic solvent comprises benzyl alcohol, benzyl benzoate, dibenzyl ether, ethyl 3-phenyl-2-propenoate, allyl 3-phenylacrylate, PEG (Mn=200-1000), PEGDA (Mn=200-1000), PEGMA (Mn=200-1000), 1-phenylnaphthalene and/or diphenyl ether.
125. The method according to any one of the preceding embodiments, wherein said clearing solution comprising the organic solvent further comprises an antioxidant.
126. The method according to any one of the preceding embodiments, wherein said clearing solution comprising the organic solvent consists of benzyl alcohol, benzyl benzoate and diphenyl ether at a volume ratio of from 4:8:3 to 10:20:3 and said antioxidant.
127. The method according to embodiment 125 or 126, wherein said antioxidant is DL-alpha-tocopherol.
128. The method according to embodiment 125 or 126 or 127, wherein said antioxidant is present in an amount of 0.4 vol % in said clearing solution.
129. The method according to any one of the preceding embodiments, wherein step d) is performed by perfusing the fixed animal tissue labeled with said fluorochrome-containing labeling agent with said clearing solution comprising the organic solvent.
130. The method according to embodiment 129, wherein the fixed animal tissue labeled with said fluorochrome-containing labeling agent is perfused with said clearing solution comprising the organic solvent for at least 6 hours.
131. The method according to embodiment 129 or 130, wherein step d) further comprises, prior to perfusion with said clearing solution, a perfusion with an increasing gradient of a dehydration solution comprising a further organic solvent of 0 vol % to 100 vol %.
132. The method according to embodiment 131, wherein said perfusion with said increasing gradient of a dehydration solution comprising said further organic solvent of 0 vol % to 100 vol % is followed by a delipidation solution comprising another organic solvent.
133. The method according to embodiment 132, wherein said further organic solvent is tert-butanol, tetrahydrofuran (THF), methanol, ethanol or 1,4-Dioxane and wherein said perfusion with said increasing gradient is performed at a temperature above the melting temperature of said further organic solvent.
134. The method according to embodiment 132 or 133, wherein said another organic solvent is dichloromethane, chloroform, methanol, hexane, butanol, ethyl acetate, tert-butyl methyl ether, and wherein said perfusion with said another organic solvent is performed at a temperature above the melting temperature of said another organic solvent.
135. The method according to any one of the preceding embodiments, wherein said labeling solution and said permeabilization solution and said clearing solution are actively delivered by applying pressure, preferably by applying pressure with a pump.
136. The method according to any one of the preceding embodiments, wherein said labeling of the target molecule with the labeling solution and said treatment with the permeabilization solution is performed by perfusion at a pressure of higher than 80 mmHg, preferably higher than 150 mmHg.
137. The method according to any one of the preceding embodiments, wherein said labeling of the target molecule with the labeling solution and said treatment with the permeabilization solution is performed by perfusion at a pressure of between 220 and 240 mmHg, preferably at a pressure of 230 mmHg.
138. The method according to any one of the preceding embodiments, wherein the fixed and decolorized animal tissue is treated with a permeabilization solution before said labeling of the target molecule in step c) and wherein the permeabilization solution and the labeling solution are different solutions.
139. The method according to embodiment 138, wherein said permeabilization solution is a dehydration solution as defined in any one of embodiments 132 or 133.
140. The method according to embodiment 138, wherein said permeabilization solution is a delipidation solution as defined in any one of embodiments 132 or 133.
141. The method according to embodiment 138, wherein said permeabilization solution comprises acetic acid.
142. The method according to embodiment 138, wherein said permeabilization solution comprises guanidine hydrochloride and/or sodium acetate.
143. The method according to any one embodiments 1 to 137, wherein the fixed animal tissue is treated with a permeabilization solution during said labeling of the target molecule in step c) and wherein the permeabilization solution and the labeling solution are the same solution.
144. The method according to any one of the preceding embodiments, wherein step b) is performed by perfusing the fixed animal tissue with said solution for the removal of heme at a pressure of higher than 80 mmHg, preferably higher than 150 mmHg.
145. The method according to any one of the preceding embodiments, wherein step b) is performed by perfusing the fixed animal tissue with said solution for the removal of heme at a pressure of between 220 and 240 mmHg, preferably at a pressure of 230 mmHg.
146. The method according to any one of embodiments 130 to 145, wherein perfusion in step d) is performed at a pressure of higher than 80 mmHg, preferably higher than 150 mmHg.
147. The method according to any one of embodiments 130 to 145, wherein perfusion in step d) is performed at a pressure of between 220 and 240 mmHg, preferably at a pressure of 230 mmHg.
148. The method according any one of the preceding embodiments, wherein said permeabilization solution comprises a cyclodextrin derivative as defined in any one of embodiments 2 to 48.
149. The method according any one of the preceding embodiments, wherein said labeling solution comprises a cyclodextrin derivative as defined in any one of embodiments 2 to 48.
150. The method according any one of the preceding embodiments, wherein the labeling solution is a composition as defined in any one of aspects 233 to 241 comprising the fluorochrome-containing labeling agent.
151. The method according to any one of the preceding embodiments, wherein said animal tissue is from a mammal.
152. The method according to any one of the preceding embodiments, wherein said animal tissue is from a non-human mammal or human.
153. The method according to any one of the preceding embodiments, wherein said animal tissue is from a rodent.
154. The method according to any one of the preceding embodiments, wherein said animal tissue is from a mouse.
155. The method according to any one of the preceding embodiments, wherein said animal tissue is a whole mouse.
156. The method according to any one of the preceding embodiments, wherein said animal tissue is pig brain.
157. The method according to any one of the preceding embodiments, wherein said animal tissue is a whole organ or a part thereof.
158. The method according to any one of the preceding embodiments, wherein said target molecule which is labeled by said labeling agent in step c) is:
   (i) a structure, preferably a protein, lipid, DNA or RNA, that is present in said fixed animal tissue, more preferably a protein that is present in said fixed animal tissue; or
   (ii) a primary antibody bound to a structure, preferably a protein, lipid, DNA or RNA, that is present in said fixed animal tissue, more preferably a protein that is present in said fixed animal tissue.
159. The method according to any one of the preceding embodiments, wherein said animal tissue contains a cancer, and wherein said target molecule which is labeled by said labeling agent in step c) is a structure, preferably a protein, lipid, DNA or RNA, that is present in said cancer, more preferably a protein that is present in said cancer.
160. The method according to any one of the preceding embodiments, wherein said animal tissue contains cancer metastases, and wherein said target molecule which is labeled by said labeling agent in step c) is a structure, preferably a protein, lipid, DNA or RNA, that is present in said cancer, more preferably a protein that is present in said cancer.
161. The method according to any one of the preceding embodiments, wherein said animal has been treated with a biopharmaceutical drug, wherein said animal tissue contains said biopharmaceutical drug, and wherein said biopharmaceutical drug is said target molecule which is labeled by said labeling agent in step c), or wherein said biopharmaceutical drug has been labeled with a further fluorochrome in vitro, or wherein said biopharmaceutical drug that is fluorescent itself.
162. The method according to embodiment 161, wherein the biopharmaceutical drug is a small molecule.
163. The method according to embodiment 161, wherein the biopharmaceutical drug is a therapeutic protein.
164. The method according to embodiment 161, wherein the biopharmaceutical drug is a therapeutic antibody.
165. The method according to any one of the preceding embodiments, wherein said method is not a method for the treatment of the human or animal body by surgery or therapy and not a diagnostic method practised on the human or animal body.
166. The method according to any one of the preceding embodiments, wherein said method is an ex vivo method.
167. The method according to any one of the preceding embodiments, wherein the animal tissue for fluorescence microscopy obtained in step d) has a smaller volume than the fixed animal tissue used in step b).
168. The method according to embodiment 167, wherein the animal tissue for fluorescence microscopy obtained in step d) has a 40% to 75% smaller volume than the fixed animal tissue used in step b).
169. The method according to any one of the preceding embodiments, further comprising, prior to labeling the target molecule in the fixed animal tissue with the labeling solution, the following step:
   c.1) contacting the fixed animal tissue with a primary antibody capable of binding to a structure, preferably a protein, lipid, DNA or RNA, that is present in said fixed animal tissue, more preferably a protein that is present in said fixed animal tissue,
      wherein the fluorochrome-containing labeling agent is capable of binding to the primary antibody.
170. The method according to embodiment 169, wherein step c. 1) is performed by perfusing the fixed animal tissue with a solution comprising the primary antibody.
171. The method according to embodiment 169 or 170, wherein step c.1) is performed by perfusing the fixed animal tissue with a primary labeling solution comprising the primary antibody, and a cyclodextrin derivative as defined in any one of embodiments 2 to 48.
172. The method according to any one of embodiments 169 to 171, wherein the primary antibody is not conjugated to a fluorochrome.
173. The method according to any one of embodiments 169 to 172, wherein the primary antibody is present in a composition as defined in any one of embodiments 233 to 241.
174. The method according to any one of embodiments 169 to 173, wherein the primary antibody is has a molecular weight as defined in any one of embodiments 80 to 97.
175. The method according to any one of embodiments 169 to 174, wherein the primary antibody w is an IgG, IgA, IgM, IgD or IgE.
176. The method according to any one of embodiments 169 to 175, wherein the primary antibody is an IgG.
177. The method according to embodiment 176, wherein the primary antibody is an IgG1, IgG2, IgG3 or IgG4.
178. The method according to any one of embodiments 169 to 177, wherein the primary antibody is a rabbit or rat antibody.
179. The method according to any one of the preceding embodiments comprising the following steps, preferably in this order:
   decolorizing, permeabilizing and blocking step performed by treating the fixed animal tissue with solution(s) for the removal of heme, permeabilization and blocking;
   labeling a target molecule in the fixed animal tissue with a labeling solution comprising the cyclodextrin derivative and the fluorochrome-containing labeling agent capable of binding to said target molecule, said labeling agent preferably having a molecular weight of more than 100 kDa, to obtain a fixed animal tissue labeled with said fluorochrome-containing labeling agent.
180. The method according to any one of the preceding embodiments comprising the following steps, preferably in this order:
   decolorizing, permeabilizing and blocking step by treating the fixed animal tissue with solution(s) for the removal of heme, permeabilization and blocking;
   contacting the fixed animal tissue with a primary labeling solution comprising the cyclodextrin derivative and a primary antibody capable of binding to a structure, preferably a protein, lipid, DNA or RNA, that is present in said fixed animal tissue, more preferably a protein that is present in said fixed animal tissue; and
   labeling the primary antibody that has bound to a structure in the fixed animal tissue with a secondary labeling solution comprising a cyclodextrin derivative and the fluorochrome-containing labeling agent capable of binding to the primary antibody, said labeling agent preferably having a molecular weight of more than 100 kDa, to obtain a fixed animal tissue labeled with said fluorochrome-containing labeling agent.
181. The method according to embodiment 179 or 180, wherein the solutions for the removal of heme, permeabilization and blocking solution are the same solution (“pretreatment solution”).
182. The method according to embodiment 181, wherein the pretreatment solution has the composition of a blocking solution as defined in any one of aspects 49 to 55.
183. The method according to any one of embodiments 169 to 182, wherein the primary labeling solution has a composition as defined in any one of aspects 233 to 241 and comprising the primary antibody.
184. The method according to any one of embodiments 169 to 183, wherein labeling with the fluorochrome-containing labeling agent is performed with a secondary labeling solution having a composition as defined in any one of aspects 233 to 241 and comprising the fluorochrome-containing labeling agent.
185. The method according to any one of embodiments 179 to 184, further comprising decalcifying the fixed animal tissue with a solution for decalcification before the decolorizing, permeabilizing and blocking step.
186. The method according to any one of embodiments 179 to 185, further comprising clearing the fixed animal tissue labeled with said fluorochrome-containing labeling agent with a clearing solution comprising an organic solvent after the labeling step(s).
187. An animal tissue or body obtainable by a method for preparing an animal tissue for fluorescence microscopy according to any one of the preceding embodiments, wherein said animal tissue contains said target molecule labeled by said fluorochrome-containing labeling agent.
188. The animal tissue of embodiment 187, wherein said animal tissue is a whole rodent, preferably a whole mouse.
189. The animal tissue of embodiment 187, wherein said animal tissue is a whole organ or a part thereof.
190. The animal tissue of embodiment 189, wherein said animal tissue is a whole mammalian organ.
191. The animal tissue of any one of embodiments 187 to 190, having a size of at least 1 mm length×at least 1 mm breadth×at least 1 mm height; preferably at least 5 mm length×at least 5 mm breadth×at least 5 mm height, more preferably at least 1 cm length×at least 1 cm breadth×at least 1 cm height.
192. The animal tissue of any one of embodiments 187 to 191, wherein said animal tissue is a tissue block of 2×2×2 cm in size.
193. The animal tissue according to any one of the preceding embodiments, wherein in said animal tissue, said target molecules are labeled by said fluorochrome-containing labeling agent, and wherein all target molecules are detectable at single-cell resolution irrespective of their location in the animal tissue when analyzed by fluorescence microscopy, preferably light-sheet fluorescence microscopy.
194. A method for analyzing an animal tissue or body according to any one of embodiments 187 to 193, said method comprising a step of i) analyzing said tissue by fluorescence microscopy in order to detect the fluorescence of said fluorochrome in said animal tissue.
195. The method for analyzing according to embodiment 194, wherein the method further comprises the step of ii) visualizing the detected fluorescence of said fluorochrome to obtain an image, preferably a three-dimensional image, of said animal tissue.
196. The method for analyzing according to embodiment 195, wherein said image is an image having single cell resolution throughout said animal tissue.
197. The method for analyzing according to any one of the preceding embodiments, wherein said animal tissue is not thicker than 20 cm, preferably not thicker than 10 cm, more preferably not thicker than 5 cm thick.
198. The method for analyzing according to any one of the preceding embodiments, wherein said animal tissue is not thicker than 2 cm and is preferably between 1.5 to 2 cm thick.
199. The method for analyzing according to any one of the preceding embodiments, wherein said method for analyzing further comprises, prior to step i), the method according to any one of embodiments 1-186.
200. The method for analyzing according to any one of the preceding embodiments, wherein said fluorescence microscopy is selected from the group consisting of light sheet fluorescence microscopy, epifluorescence microscopy, multi-photon microscopy and confocal fluorescence microscopy.
201. The method for analyzing according to any one of the preceding embodiments, wherein said fluorescence microscopy is fluorescence microscopy, preferably light sheet fluorescence microscopy.
202. The method for analyzing according to any one of the preceding embodiments, wherein said method further comprises, after step i), the steps of iii) dissecting a tissue region of interest; iv) rehydrating the dissected tissue region of interest; and v) further analyzing the dissected tissue region of interest.
203. The method for analyzing according to embodiment 202, wherein in step v), the dissected tissue region of interest is further analyzed by antibody-based immunostaining, or by gene-profiling which is preferably gene profiling by RNAseq, or by proteomics which is preferably proteomics by mass spectrometry.
204. The method for analyzing according to any one of embodiments 202 or 203, wherein the dissected tissue region of interest comprises a metastasis, preferably a metastasis having a size of less than 200 tumor cells, more preferably a metastasis having a size of less than 100 tumor cells, still more preferably a metastasis having a size of less than 75 tumor cells, still more preferably a metastasis having a size of less than 50 tumor cells, and still more preferably a metastasis having a size of less than 25 tumor cells.
205. A method for the detection of metastases, wherein the method comprises a method for analyzing an animal tissue according to any one of the preceding embodiments.
206. The method for the detection of metastases according to embodiment 205, which is a method for the detection of metastases in said animal tissue at single-cell resolution throughout said animal tissue.
207. The method for the detection of metastases according to any one of the preceding embodiments, wherein said animal tissue contains cancer metastases, and wherein the target molecule labeled by said labeling agent is a structure, preferably a protein, that is present in said cancer.
208. A method for analyzing the biodistribution of a biopharmaceutical drug, wherein the method comprises a method for analyzing an animal tissue according to any one of the preceding embodiments, wherein said animal has been treated with the biopharmaceutical drug as defined in embodiment 161, wherein said animal tissue contains said biopharmaceutical drug, and wherein said biopharmaceutical drug is said target molecule which is labeled by said labeling agent.
209. The method according to embodiment 208, wherein the biopharmaceutical drug is a therapeutic protein, preferably a therapeutic antibody.
210. The method according to embodiment 208, wherein the biopharmaceutical drug is a nanoparticle.
211. A method for analyzing the biodistribution of nanoparticles, wherein the method comprises a method for analyzing an animal tissue according to any one of the preceding embodiments, wherein said animal has been treated with the nanoparticles, wherein said animal tissue contains said nanoparticles, and wherein said nanoparticles are said target molecule which is labeled by said labeling agent and/or are selected from fluorochrome-conjugated nanoparticles or nanoparticles which are fluorescent themselves.
212. The method for analyzing the biodistribution of nanoparticles according to embodiment 211, wherein the nanoparticles carry a drug or are a drug.
213. A method for studying neurodegeneration, wherein the method comprises a method for analyzing an animal tissue according to any one of the preceding embodiments, and wherein said animal tissue contains neurons.
214. The method for studying neurodegeneration according to embodiment 213, wherein the neurons in said animal tissue have been fluorescently labeled, preferably by expression of a fluorescent protein.
215. The method for studying neurodegeneration according to any one of the preceding embodiments, wherein the studying of neurodegeneration comprises the analysis of the blebbing of neuronal axons.
216. A method for studying neuroinflammation, wherein the method comprises a method for analyzing an animal tissue according to any one of the preceding embodiments, and wherein said animal tissue contains neurons.
217. The method for studying neuroinflammation according to embodiment 216, wherein immune cells in said animal tissue have been fluorescently labeled, preferably by expression of a fluorescent protein.
218. The method for studying neuroinflammation according to any one of the preceding embodiments, comprising studying the activation of immune cells by analyzing the signal intensity and/or the cell numbers of the fluorescently labeled immune cells.
219. A method for studying meningeal lymphatic vessels, wherein the method comprises a method for analyzing an animal tissue according to any one of the preceding embodiments, and wherein said animal tissue comprises preferably an intact mouse head.
220. The method for studying meningeal lymphatic vessels according to embodiment 219, wherein said meningeal lymphatic vessels are fluorescently labeled, preferably by a marker protein or by a tracer such as ovalbumin,
221. The method for studying meningeal lymphatic vessels according to any one of the preceding embodiments, wherein said meningeal lymphatic vessels contain said target molecule.
222. Use of a cyclodextrin derivative for improving labeling of a target molecule in a fixed animal tissue or whole animal body with a fluorochrome-containing labeling agent.
223. The use according to embodiment 222, wherein the fluorochrome-containing labeling agent is a fluorescent antibody.
224. The use according to embodiment 222 or 223, wherein the fluorochrome-containing labeling agent has a molecular weight of more than 100 kDa.
225. The use according to any one of embodiments 222 to 224, wherein the fluorochrome-containing labeling agent is as defined in any one of embodiments 80 to 115.
226. The use according to any one of embodiments 222 to 225, wherein the cyclodextrin derivative is as defined in any one of embodiments 2 to 48.
227. The use according to any one of embodiments 222 to 226, wherein the cyclodextrin derivative is not methyl-β-cyclodextrin with a substitution degree of 1.8.
228. The use according to any one of embodiments 222 to 227, wherein the cyclodextrin derivative is not methyl-β-cyclodextrin with a substitution degree of <2.0.
229. The use according to any one of embodiments 226 to 228, with the proviso that when R², R³ and R⁶ are each selected from H and CH₃, then DS≥2.
230. The use according to any one of embodiments 226 to 229, wherein:
   R² and R⁶ are CH₃, and R³ is H; or
   R², R³ and R⁶ are each independently selected from H and CH₂CH(OH)CH₃, and/or DS≥0.9
231. The use according to any one of embodiments 222 to 229, wherein the cyclodextrin derivative is selected from (2-hydroxypropyl)-β-cyclodextrin, triacetyl-β-cyclodextrin, (2-hydroxyethyl)-β-cyclodextrin, heptakis (2,6-di-O-methyl)-β-cyclodextrin, succinyl-β-cyclodextrin γ-cyclodextrin and α-cyclodextrin; more preferably (2-hydroxypropyl)-β-cyclodextrin or heptakis (2,6-di-O-methyl)-β-cyclodextrin; most preferably heptakis (2,6-di-O-methyl)-β-cyclodextrin.
232. The use according to any one of embodiments 222 to 231, wherein the use is not a method for the treatment of the human or animal body by surgery or therapy, and is not a diagnostic method practised on the human or animal body.
233. Composition comprising an antibody, preferably having a molecular weight of more than 100 kDa, and a cyclodextrin derivative.
234. The composition according to embodiment 233, wherein the cyclodextrin derivative is as defined in any one of embodiments 2 to 48.
235. The composition according to embodiment 234, with the proviso that when R², R³ and R⁶ are each selected from H and CH₃, then DS≥2.
236. The composition according to embodiment 233 or 234, wherein:
   R² and R⁶ are CH₃, and R³ is H; or
   R², R³ and R⁶ are each independently selected from H and CH₂CH(OH)CH₃, and/or DS≥0.9
237. The composition according to any one of embodiments 233 to 236, wherein the antibody is a fluorochrome-containing labeling agent, preferably as defined in any one of embodiments 80 to 115.
238. The composition according to any one of embodiments 233 to 236, wherein the antibody is a primary antibody, preferably as defined in any one of embodiments 172 to 178.
239. The composition according to any one of embodiments 233 to 238, further comprising one or more, preferably all of the following components, in aqueous buffer solution, preferably phosphate buffer saline (PBS):
   animal serum, preferably mammalian serum, more preferably goat serum;
   zwitterionic surfactant, preferably CHAPS or CHAPSO, more preferably CHAPS;
   non-ionic surfactant, preferably Triton X-100 or IGEPAL CA-630, more preferably Triton X-100;
   organic solvent, preferably a water-miscible solvent, more preferably DMSO;
   amino acid, preferably glycine.
240. The composition according to embodiment 239, wherein the components, if present in the aqueous buffer solution, have the following concentrations:
   cyclodextrin derivative: 0.5 to 2, preferably 0.75 to 1.5, more preferably 1% w/v;
   animal serum: 0.5 to 12, preferably 0.75 to 10, more preferably 1 to 3% v/v;
   zwitterionic surfactant: 5 to 15, preferably 7.5 to 12.5, more preferably 10% w/v;
   non-ionic surfactant: 0.5 to 4, preferably 1 to 3, more preferably 2% w/v;
   organic solvent: 5 to 20, preferably 7.5 to 15, more preferably 10% w/v;
   amino acid: 0.5 to 2, preferably, 0.75 to 1.5, more preferably 1% w/v;
   and wherein the aqueous buffer has a buffer agent concentration of 0.05 to 0.2 M, preferably 0.08 to 1.2 M, more preferably 0.1 M.
241. The composition according to any one of embodiments 233 to 240, comprising:
   0.5 to 2, preferably 0.75 to 1.5, more preferably 1% w/v cyclodextrin derivative,
   0.5 to 12, preferably 0.75 to 10, more preferably 1 to 3% v/v goat serum;
   5 to 15, preferably 7.5 to 12.5, more preferably 10% w/v CHAPS;
   0.5 to 4, preferably 1 to 3, more preferably 2% w/v Triton X-100;
   5 to 20, preferably 7.5 to 15, more preferably 10% w/v DMSO; and
   0.5 to 2, preferably, 0.75 to 1.5, more preferably 1% w/v glycine;
   in 0.05 to 0.2 M, preferably 0.08 to 1.2 M, more preferably 0.1 M phosphate buffer saline.

### BRIEF DESCRIPTION OF THE DRAWINGS

FIG. 1: Development of wildDISCO and single antibody whole-mouse staining. (a) The structure of cyclodextrin (CD) with different substituent groups, CD1 (Methyl-β-cyclodextrin), CD2 (2-Hydroxypropyl-β-Cyclodextrin), CD3 (Triacetyl-β-cyclodextrin), CD4 ((2-Hydroxyethyl)-β-cyclodextrin), CD5 (Heptakis (2,6-di-O-methyl)-β-cyclodextrin), CD6 (Succinyl-β-cyclodextrin). (b) Measurements of supernatant cholesterol concentration after different CD-containing buffer incubation at 7th day for 25 mg mouse liver sections with control, CD1, CD2, CD4, CD5, CD6, γ-cyclodextrin (CD7) and α-cyclodextrin (CD8). (c) Methylene blue staining of single hemisphere of mouse brains after permeabilization with different CD-containing solutions. CD5 is shown to greatly enhance tissue permeabilization for dye migration compared to others. (d) Dynamic light scattering (DLS) for size distribution of TH antibody in solutions with and without CD5. (e) Deep color coding shows the pan-neuronal mark-er PGP-9.5+ neuronal projections at different z-levels in the 2.0 cm-thick whole mouse body. (f,g) Details of innervation throughout hard (f, vertebrae) and soft tissues (g, adipose tissue). (h) Manually segmented vagus nerves (green) innervating the liver (cyan), spleen (magenta), intestine (red) and kidney (yellow), highlighted with specific pseudo-colors. (i) A whole mouse stained with a lymphatic vessel marker LYVE1 (yellow). (j) Lymphoid elements (LYVE1) staining was detected in the brain parenchyma of (i) mouse. (k) Mouse brains stained with two different lymphatic vessel markers (LYVE1 and podoplanin) to identify lymphatic endothelial cells were found in the different brain regions.

FIG. 2: Studying the spatial relationship of different physiological systems using wildDISCO. (a) Maximum intensity projection of a mouse stained with antibodies against the sympathetic nerve marker tyrosine hydroxylase (TH) (green) and the immune cell marker CD45 (magenta), showing the landscape of neuro-immune interactions in internal organs. (b) The branches of the sympathetic nervous system (TH, green) connect different regions of the intestine. CD45+ cells (magenta) accumulate along parts of the vagus nerve, especially at the inferior mesenteric plexus. (c) High-magnification views of the labeled regions in (a), showing the colocalization of the sympathetic nerve fibers and immune cells on the intestinal wall. (d) Maximum intensity projections of a whole mouse stained with TH (green) and LYVE1 (yellow). (e-g) Representative 2D optical sections of hindlimb LNs stained with TH, PGP 9.5, CD45, Prox1 and LYVE1 as indicated in the images to show the LNs are innervated by peripheral nerves with immunomodulatory potential. (h,i) Representative 3D representation of the myenteric nerve lattice net-work of WT mice and germ-free mice by immunostaining with antibodies against PGP9.5. (j.k) Higher magnification views of the regions marked by the blue and red boxes in (h) and (j), respectively. In the germ-free mice, the myenteric nerve lattice network appears disorganized, with fewer ganglia. (l) The density of PGP 9.5 myenteric plexus was quantified. n=5; mean±SD; **p<0.01 (Student's t test).

FIG. 3: Overview of wildDISCO immunostaining buffer and quantification of permeabilization efficacy.
(a) Illustration of primary chemical components involved in the wildDISCO immunostaining buffer.
(b) Profile plot along each mouse brain dimension in FIG. 1c.

FIG. 4: wildDISCO immunostaining of PGP9.5 in the whole mouse body. (a) Maximum projection of peripheral nerve system from a 4-week-old mouse stained with PGP 9.5 antibody using light sheet microscopy. (b-e) Examples of positive PGP 9.5 staining in various organs (heart, spleen, liver, and intestine) with higher magnified areas. (f) Visualization of peripheral nerve innervation on multiple organs (adrenal gland (green), kidney (magenta), and ureter (cyan)).

FIG. 5: wildDISCO immunostaining of LYVE1 in the whole mouse body. Optical sections examples of whole mouse staining with LYVE1 antibody in different organs, liver (a), hindlimb (b), adipose tissue (c), kidney (d), trachea (e),stomach (f), intestine (g) with higher magnified regions. (h-i) 3D reconstruction view of the intestinal lymphatic network using Syglass reconstruction software.

FIG. 6: Nerve-immune cell interactions in the gut.
(a) 3D reconstruction view of a Peyer's patch in the intestine stained with CD45 (green) and innervated by TH+ sympathetic nerve (magenta), visualized with Syglass software. (b) Sympathetic nerve marker TH (green) and immune cells marker CD45 (magenta) on the intestinal wall, showed with Imaris software.

FIG. 7: Nerve-lymphatic vessel interactions in the gut. (a) PGP9.5 nerve fibers (magenta) interacted with Prox1 lymphatic vessel (green). (b) TH sympathetic nerve (green) and LYVE1 lymphatic vessel (magenta).

FIG. 8: Nerve-lymphatic vessel interactions in the kidney. (a) The TH sympathetic neuron (green) innervated the LYVE1 lymphatic vessel (magenta) in the kidney. (b) The PGP 9.5 pan-neuronal marker (green) combined with Prox1 lymphatic vessel marker (magenta).

FIG. 9: Influence of the microbiota on sympathetic nerve of the mice. (a-b) The myenteric nerve lattice network of WT mice and germ-free mice by immunostaining with antibodies against TH. Higher magnification views of the regions marked by the white and yellow boxes, respectively.

FIG. 10: wildDISCO applied into cancer metastasis model. Tyrosine hydroxylase TH was co-stained with breast cancer cells MDA-MB-231. The cancer cells were clearly visible closely around the TH+ nerves.

FIG. 11: wildDISCO reveals whole mouse artery networks. Alpha Smooth Muscle Actin (alpha-SMA) was applied to investigate the distribution of arteries at the whole-body scale. The larger-diameter arteries branch into arterioles, penetrating from the meninges to the corpus callosum (A). The distribution of arterioles was symmetrical and regular in the eye, olfactory bulb, meninges, and cerebrum (B). As alpha SMA is the defining hallmark of mature cardiac fibroblast, termed myofibroblasts, the coronary artery and its associated branches were clearly visualized in the heart (C). Alpha SMA was detected in the hepatic sinusoidal space as well as the portal and central veins (D). The splenic artery branched into arterioles that shaped the reticular capillary network. (E) The panicle-like vascular architecture was also observed in kidneys and lungs (F and G). The cross-section of dorsal alpha-SMA labeled mice revealed the distribution of arteries in several organs, especially the arteriole in the spinal cord (H).

### DETAILED DESCRIPTION OF THE INVENTION

### Definitions and General Techniques

Unless otherwise defined below, the terms used in the present invention shall be understood in accordance with their common meaning known to the person skilled in the art.

All publications, patents and patent applications cited herein are hereby incorporated by reference in their entirety for all purposes. These publications, patents and patent applications referred to herein are preferably identified by the name of their first author and the year of publication, or by a number. For each of the references which are identified in this way, the respective corresponding reference including the specific source of the publication (e.g. the name and the volume of the scientific journal, etc.) can be found in the section entitled “references”.

The materials, methods, and examples are illustrative only and are not intended to be limiting, unless otherwise specified.

The term “fluorochrome” as used herein is not particularly limited. For example, the fluorochrome may be a fluorescent protein or a synthetic compound such as a synthetic organic compound. Preferably, fluorochromes used according to the invention are capable of emitting fluorescence in the red or infrared range, more preferably in the far-red or near-infrared range. Preferred wavelengths for the emission maximum of fluorochromes used according to the invention are as indicated in the preferred embodiments of the present invention. Non-limiting examples of fluorochromes which are capable of emitting fluorescence in the far-red or near-infrared range, and which can be used in accordance with the present invention, are known in the art and have been reviewed, for example, in Hong et al (2017), Near-infrared fluorophores for biomedical imaging. Nature Biomedical Engineering 1, 0010, which is incorporated by reference in its entirety for all purposes. Fluorochromes which are capable of emitting fluorescence in the far-red or near-infrared range, and which can be used in accordance with the present invention, are commercially available and preferably include, for instance, ATTO dyes such as ATTO Rho13, ATTO 594, ATTO 550, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, and ATTO 740 and Alexa Fluor® dyes such as Alexa Fluor® 568, Alexa Fluor® 594, Alexa Fluor® 610, Alexa Fluor® 633, Alexa Fluor® 635, Alexa Fluor® 647, Alexa Fluor® 660, Alexa Fluor® 680, Alexa Fluor® 700, Alexa Fluor® 750 and Alexa Fluor® 790, and Cy dyes such as Cy7, Cy5 and Cy3. Dyes with emission maxima at 488 nm, 555 nm and 568 nm are also known in the art and can also be used in the methods of the invention.

Labeling with fluorochrome-containing labeling agents that have a molecular weight of equal to or less than 100 kDa is also contemplated. That is, while the present invention is particularly advantageous for improving or improving labeling of a target molecule in a fixed animal tissue or whole animal body with fluorescent labeling agents (such as antibodies) having a molecular weight of more than 100 kDa, it is also contemplated that comparable improvements can be achieved by using the cyclodextrin derivatives described herein for improving labeling with agents that have a molecular weight of equal to or less than 100 kDa (e.g., antibody fragments conjugated to a fluorochrome, or other dyes).

The animal tissues that can be used in the uses and methods of the present invention are not particularly limited. They can be from any animal species. In preferred embodiments of the present invention, the animal tissue can be a tissue from a non-human mammal or from a human. Preferably, the animal tissue from the non-human mammal is from a rodent, more preferably from a mouse. Still more preferably, the animal tissue is a whole mouse. In preferred embodiments in accordance with the invention, the animal tissue can be a whole organ or a part thereof, preferably a human organ or part thereof. The animal tissue may contain recombinantly expressed fluorescent proteins (e.g. GFP, YFP and mCherry), which can be used as target molecules. For example, the animal from which the animal tissue has been obtained can be an animal (e.g. a mouse) that has been transplanted with cancer cells expressing such recombinant fluorescent proteins. The fixed animal tissue used in the uses and methods of the present invention has preferably a size of at least 1 cm length×at least 1 cm breadth×at least 1 cm height.

The decolorizing step of the methods for preparing of the invention uses a fixed animal tissue. In preferred embodiments in accordance with the invention, the method for preparing of the invention starts with the decolorizing step and does not comprise the fixation of the animal tissue. Accordingly, in preferred embodiments, all methods and uses of the invention can preferably be a method or use which is not a method for the treatment of the human or animal body by surgery or therapy and not a diagnostic method practised on the human or animal body. In related preferred embodiments in accordance with all other embodiments of the invention, the methods or uses of the invention are ex vivo methods and uses. Thus, the methods of the invention can preferably be carried out outside a living animal.

Fixed animal tissues which are suitable for the methods of the invention can readily be identified by a person skilled in the art. For example, for PFA fixation, mice can be deeply anesthetized using a combination of midazolam, medetomidine and fentanyl (MMF) (e.g. 1 mL/100 g of body mass for mice; i.p.) before intracardial perfusion with heparinized 0.1 M PBS (10 U/mL of Heparin, Ratiopharm; 100-125 mmHg pressure using a Leica Perfusion One system) for 5-10 minutes at room temperature until the blood is washed out. This procedure can be followed by fixation, e.g. with 4% paraformaldehyde (PFA), for instance in 0.1 M PBS (pH 7.4) (Morphisto, 11762.01000) for 10-20 minutes. If vasculature staining is desired, the animal tissues such as mice (e.g. whole mice) can be intracardially perfused with 20 ml of PBS (without Heparin) containing 0.5 mg of FITC-conjugated Lectin (EY Laboratories, F-2101-5), before proceeding with PFA fixation. Alternatively, for PaXgene fixation, the mice can be deeply anesthetized using a combination of midazolam, medetomidine and fentanyl (MMF) (e.g. 1 mL/100 g of body mass for mice; i.p.) before intracardial perfusion with heparinized 0.1 M PBS (10 U/mL of Heparin, Ratiopharm; 100-125 mmHg pressure using a Leica Perfusion One system) for 5-10 minutes at room temperature until the blood is washed out. This procedure can be followed by fixation via injection of 40-50 ml of Paxgene fixation solution. If storage of Paxgene fixed animal is needed before further processing, the tissues are kept in Paxgene stable solution. If vasculature staining is desired, the animal tissues such as mice (e.g. whole mice) can be intracardially perfused with 20 ml of PBS (without Heparin) containing 0.5 mg of FITC-conjugated Lectin (EY Laboratories, F-2101-5), before proceeding with PFA fixation. Subsequently, the skin can be carefully removed or left intact if the animal is nude (no furs) and the bodies can be post-fixed in 4% PFA for 1 day at 4° C. and transferred to 0.1 M PBS. The method of the invention can be started immediately or whole mouse bodies can be stored, preferably in PBS at 4° C. for up to 4 weeks or in PBS containing 0.05% sodium azide (Sigma, 71290) for up to 6 months.

As used herein, terms such as “an animal tissue for fluorescence microscopy” are meant to indicate that the respective animal tissue is suitable for fluorescence microscopy.

Similarly, the term “for the removal of heme” in connection with a solution refers to any solution that is suitable for the removal of heme. In accordance with the invention, the removal of heme is not limited to a particular mechanism as long as it removes heme from the tissue and/or decolorizes the heme. For example, aminoalcohols suitable for the removal of heme such as N,N,N′,N′-tetrakis (2-hydroxypropyl)ethylenediamine can be used, e.g. as indicated in the preferred embodiments. Such aminoalcohols compete with hemoglobin for heme binding and can thus be used to remove heme from the hemoglobin in the tissue and from the tissue. Alternatively, benzyl peroxide can be used.

The term “fluorochrome-containing labeling agent” as used in accordance with the invention is not particularly limited as long as it is suitable to label target molecules in the decolorized fixed animal tissue, is capable of binding to said target molecule and has a molecular weight of more than 100 kDa (e.g., equal to or more than 110 kDa, equal to or more than 120 kDa, equal to or more than 130 kDa or equal to or more than 140 kDa). In preferred embodiments, the fluorochrome-containing labeling agent is an antibody conjugated to said fluorochrome.

As used herein, the term “target molecule” refers to any target molecule in the tissue. It is understood that for a given application of the methods of the invention such as biomedical applications, appropriate target molecules can be selected. These target molecules can, for instance, be endogenous molecules of the animal (e.g. marker proteins for diseases such as cancer) or recombinant molecules such as recombinant proteins. For example, in preferred embodiments in accordance with the invention, if the animal from which the animal tissue has been obtained is an animal (e.g. a mouse) that has been transplanted with cancer cells expressing such recombinant fluorescent proteins, such a fluorescent protein can be a target molecule. Alternatively, the target molecule may be any other structure (e.g., an exogenous molecule) that is present in the fixed animal tissue, for example, a protein, lipid, DNA or RNA, that is present in said fixed animal tissue, more preferably a protein that is present in said fixed animal tissue, e.g., a therapeutic antibody, or a primary antibody that has bound to a tissue antigen in the fixed animal tissue.

Primary antibodies that can be used in the present invention are not particularly limited. In one embodiment, a primary antibody is selected from the group consisting of an anti-Tyrosine hydroxylase antibody, an anti-PGP 9.5 antibody, an anti-S100 beta antibody, an anti-Neurofilament M antibody, an anti-Alpha smooth muscle actin antibody, an anti-Collagen IV antibody, an anti-Prox1 antibody, an anti-LYVE1 antibody, an anti-Iba1 antibody, and an anti-CD45 antibody. Preferably, the primary antibody is not a rabbit anti-neurofilament NF-M antibody.

It is also to be understood that the term “labeling a target molecule” as used herein includes the possibility that more than one target molecule can be labeled by the methods of the invention. Thus, in preferred embodiments in accordance with the invention, more than one target molecule is labeled, e.g. two or three target molecules. For example, in preferred embodiments in accordance with the invention, if the animal from which the animal tissue has been obtained is an animal (e.g. a mouse) that has been transplanted with cancer cells expressing such recombinant fluorescent proteins, such a fluorescent protein can be first target molecule, and a biopharmaceutical drug against cancer (e.g. a therapeutic antibody against cancer) that has been administered to said animal can be a second target molecule. Thus, in preferred embodiments such as the present embodiment, the method for the detection of metastases according to the invention and the method for analyzing the biodistribution of a biopharmaceutical drug can be carried out together.

As used herein, the term “perfusion at a pressure of” refers to the pressure which is measurable at the entry point of the tissue. The pressure can be measured by any methods known in the art. Preferably, the pressure is measured with an manometer, more preferably with a Kkmoon Digital Manometer Pressure Gauge Manometer (HT-1891). When using the Kkmoon Digital Manometer Pressure Gauge Manometer (HT-1891), a 2 heads connector (B.Braun Discofix® C Dreiwegehahn, 16494C) can be inserted to the pumping channel and connected to the manometer. The pumping channel can be set with transcardiac perfusion needle (Leica, 39471024) and the pressure can be measured (when the readouts are stable) at the pumping speed used for the method.

A “permeabilization solution” as referred to herein means a solution that is suitable for permeabilization of the animal tissue. Such solutions are known in the art and can readily be selected by a person skilled in the art and include, for instance, suitable surfactants for permeabilization such as CHAPS (3-[(3-cholamidopropyl)-dimethylammonio]-1-propanesulfonate; CAS Number: 75621-03-3), CHAPSO (3-([3-cholamidopropyl]dimethylammonio)-2-hydroxy-1-propanesulfonate; CAS Number: 82473-24-3), IGEPAL CA-630 (octylphenoxy poly(ethyleneoxy) ethanol; CAS Number: 68412-54-4) and/or Triton X-100 (4-(1,1,3,3-tetramethylbutyl)phenyl-polyethylene glycol; CAS Number: 9002-93-1).

A “labeling solution” as referred to herein means a solution comprising the fluorochrome-containing labeling agent, unless stated otherwise. Typically, the labeling solution comprises one or more surfactants, an organic solvent, and the labeling agent. Advantageously, the labeling solution comprises one or more, preferably all of the following components, in aqueous buffer solution, preferably phosphate buffer saline (PBS): animal serum, preferably mammalian serum, more preferably goat serum; zwitterionic surfactant, preferably CHAPS or CHAPSO, more preferably CHAPS; non-ionic surfactant, preferably Triton X-100 or IGEPAL CA-630, more preferably Triton X-100; organic solvent, preferably a water-miscible solvent, more preferably DMSO; and/or amino acid, preferably glycine.

Preferably, the permeabilization solution and/or the labeling solution used in accordance with the invention further comprises an agent suitable to extract the cholesterol from biological membranes. Without wishing to be bound by any theory, it is hypothesized that poor cholesterol extraction from cell membranes might have been the limiting factor for permeabilization in prior art protocols.¹⁸ Agents suitable to extract the cholesterol from biological membranes include, for instance, cyclodextrin derivatives. Preferred cyclodextrin derivatives are summarized in Table 1 below.

TABLE 1
Cyclodextrin derivatives

| | | | | Average degree of |
|---|---|---|---|---|
| | | | | substitution (DS) |
| | | | | per glucopyranose |
| | Structrure | R², R³, R⁶ | Name | unit |
| CD1 | 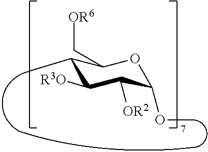 | H or CH₃ | Methyl-β-cyclodextrin | 1.8 |
| CD2 | 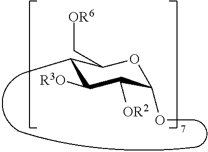 | H or CH₂CH(OH)CH₃ | (2-Hydroxypropyl)-β- cyclodextrin | 0.9 |
| CD3 | 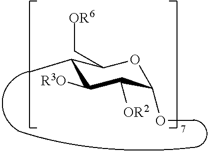 | H or C(O)CH₃ | Triacetyl-β- cyclodextrin | 3.0 |
| CD4 | 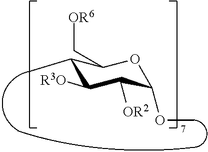 | H or CH₂CH₂OH | (2-Hydroxyethyl)-β- cyclodextrin | 0.7 |
| CD5 | 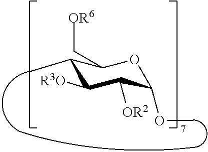 | R², R⁶ = CH₃; R³ = H | Heptakis(2,6-di-O- methyl)-β-cyclodextrin | 2.0 |
| CD6 | 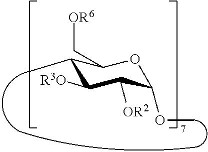 | C(O)CH₂CH₂C(O)OH | Succinyl-β- cyclodextrin | 0.5 |
| CD7 | 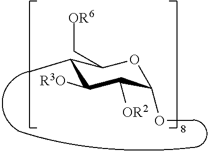 | H | γ-Cyclodextrin | 0.0 |
| CD8 | 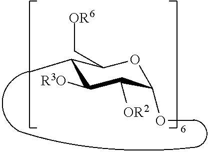 | H | α-Cyclodextrin | 0.0 |

As used herein, the term “total degree of substitution” or “TDS” refers to the total molar average number of non-hydrogen substituents R², R³, R⁶ per mol of substituted cyclodextrin. TDS can be determined by methods known in the art, such as ¹H NMR, ESI-MS or MALDI-TOF-MS. For a certain substituted cyclodextrin wherein the non-hydrogen substituents are represented by R, TDS can be calculated based on the number average molecular weight, Mₙ, according to the following formula:

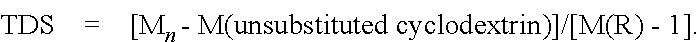

As used herein, the term “degree of substitution” or “DS” refers to the molar average number of non-hydrogen substituents R², R³, R⁶ per mol of glucopyranose unit of the cyclodextrin. DS can be determined by methods known in the art. For a certain substituted cyclodextrin, DS can be calculated based on the total degree of substitution (TDS), as defined above, according to the following formula:

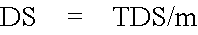

wherein m is the number of glucopyranose units in the cyclodextrin molecule (m=6 for α-cyclodextrins; m=7 for β-cyclodextrins; m=8 for γ-cyclodextrins).

Preferred cyclodextrin derivatives are those wherein m=7, i.e., β-cyclodextrins. The cyclodextrin derivatives are preferably selected from (2-hydroxypropyl)-β-cyclodextrin, triacetyl-β-cyclodextrin, (2-hydroxyethyl)-β-cyclodextrin, heptakis (2,6-di-O-methyl)-β-cyclodextrin, succinyl-β-cyclodextrin γ-cyclodextrin and α-cyclodextrin; more preferably (2-hydroxypropyl)-β-cyclodextrin or heptakis (2,6-di-O-methyl)-β-cyclodextrin; most preferably heptakis (2,6-di-O-methyl)-β-cyclodextrin. The cyclodextrin derivative is preferably not methyl-β-cyclodextrin, e.g., it is not methyl-β-cyclodextrin with a substitution degree of <2.0, e.g., 1.8. Particularly preferred cyclodextrin derivatives are those wherein R², R⁶═CH₃ and R³═H, more preferably heptakis (2,6-di-O-methyl)-β-cyclodextrin (i.e., CD5); or wherein R², R³ and R⁶ are selected from H and CH₂CH(OH)CH₃, more preferably (2-hydroxypropyl)-β-cyclodextrin (e.g., CD2); most preferably heptakis (2,6-di-O-methyl)-β-cyclodextrin.

Preferably, the cyclodextrin derivatives used in the present invention are capable of extracting cholesterol from mouse liver tissue such that the cholesterol concentration is ≥14, preferably ≥30, more preferably ≥50, most preferably >70 UM after 7 days of incubation, measured under the conditions described under “Screening of cyclodextrin-containing buffer for cholesterol extraction” in the Examples below.

Preferably, the permeabilization solution used in accordance with the invention further comprises an agent to loosen the collagen network, e.g. trans-1-acetyl-4-hydroxy-L-proline. It is to be understood that if a permeabilization solution is used in a particular step of the methods of the invention, this does not exclude that other solutions, e.g. solutions used in prior steps of the method may also contribute to the permeabilization and improve permeabilization. For instance, the solution for the removal of heme can be a solution that contributes to the permeabilization.

Measurements of tissue volumes according to the invention can be made by any suitable methods known in the art. Preferably, such volumes are measured by measuring the volume displacement of liquids by the tissue, e.g. in a suitable cylinder.

Preferably, the method of the invention further comprises a blocking step. The blocking step is performed by treating the fixed animal tissue with a blocking solution before the step of labeling. A “blocking solution” as referred to herein is suitable for blocking unspecific antigen binding of antibodies and can comprises, e.g., an animal serum. The animal serum can be mammalian serum, preferably goat serum or donkey serum, more preferably goat serum; and can be present, e.g., at a concentration of 1 to 15, preferably 3 to 10, more preferably 3% v/v. The blocking solution can further comprise a surfactant, e.g., a non-ionic surfactant, preferably Triton X-100 or IGEPAL CA-630, more preferably Triton X-100. The non-ionic surfactant can be present, e.g., at a concentration of 0.5 to 4, preferably 1 to 3, more preferably 2% w/v. The components of the blocking solution are preferably present in an aqueous buffer solution, preferably phosphate buffer saline (PBS), having buffer agent concentration of 0.05 to 0.2 M, preferably 0.08 to 1.2 M, more preferably 0.1 M.

In a preferred embodiment, the treating with permeabilization solution is performed simultaneously with the treating with blocking solution, e.g., by providing a permeabilization solution which at the same time is the blocking solution, preferably comprising animal serum and surfactant as described above.

A particularly preferred method according to the present invention comprises the following steps, preferably in this order:
decolorizing, permeabilizing and blocking step performed by treating the fixed animal tissue with solution(s) for the removal of heme, permeabilization and blocking;
labeling a target molecule in the fixed animal tissue with a labeling solution comprising the cyclodextrin derivative and the fluorochrome-containing labeling agent capable of binding to said target molecule, said labeling agent preferably having a molecular weight of more than 100 kDa, to obtain a fixed animal tissue labeled with said fluorochrome-containing labeling agent.

An even more preferred method according to the present invention comprises the following steps, preferably in this order:
decolorizing, permeabilizing and blocking step performed by treating the fixed animal tissue with solution(s) for the removal of heme, permeabilization and blocking;
contacting the fixed animal tissue with a primary labeling solution comprising a cyclodextrin derivative and a primary antibody capable of binding to a structure, preferably a protein, lipid, DNA or RNA, that is present in said fixed animal tissue, more preferably a protein that is present in said fixed animal tissue,
labeling the primary antibody that has bound to a structure in the fixed animal tissue with a secondary labeling solution comprising the cyclodextrin derivative and the fluorochrome-containing labeling agent said labeling agent being is capable of binding to the primary antibody, and preferably having a molecular weight of more than 100 kDa, to obtain a fixed animal tissue labeled with said fluorochrome-containing labeling agent.

The solution for the removal of heme, permeabilization and blocking solution are preferably the same solution (“pretreatment solution”), and preferably has the composition of a blocking solution as described hereinabove. The primary and secondary labeling solutions preferably have the composition of a labeling solution as described hereinabove.

A “clearing solution” as referred to herein means a solution that is suitable for the clearing of the animal tissue. Such solutions are not particularly limited as long as they contain an organic solvent. It is understood that such organic solvents can readily be selected by a skilled person such that they are compatible with the methods of the invention, e.g. based on their electromagnetic absorption/emission spectra (in particular their lack of fluorescent emission in the visible, red and near-infrared range). Preferably, the clearing solution comprising the organic solvent has a refractive index that is similar to the tissue (e.g. the bones) of said animal, as reflected in the preferred embodiments. Such clearing solutions are particularly advantageous for the clearing of the tissue. Examples of preferred organic solvents that can be used in such clearing solutions of the invention are, for instance, a solvent comprising benzyl alcohol, benzyl benzoate and diphenyl ether, a solvent comprising ethyl cinnamate and a solvent comprising allyl cinnamate.

Methods for measuring the refractive index are well-known in the art. The refractive index values referred to herein are values which have been measured at room temperature (i.e. 25° C.) and normal atmospheric pressure (i.e. 760 mmHg).

A “further organic solvent” as referred to in connection with the present invention is not particularly limited. It is understood that such solvent will be selected by the skilled person such that they are suitable for dehydration. Examples of such solvents are THF, dichloromethane and 1,4-Dioxane. For example, according to the invention, a preferred perfusion with an increasing gradient of a further organic solvent of 0 vol % to 100 vol % can be a perfusion with a gradient of 0 vol % to 100 vol % THF, followed by an incubation with dichloromethane. Alternatively, in all embodiments in accordance with the invention, dichloromethane can be replaced by 1,4-Dioxane.

An additional improvement in accordance with all other embodiments of the invention can be achieved by the addition of a decalcification step. Such a step will further improve the clearing of bones. Decalcification chemicals are known and include, for example, solutions comprising EDTA, preferably further comprising NaHCO₃. Such a decalcification step is performed prior to the decolorization step, or if no decolorization step is performed, prior to the labeling step.

The term “present in said fixed animal tissue” as used herein in connection with structures refers to structures which are present in said fixed animal tissue. This term does not mean that the structure must be present inside a cell but also includes the possibility that the structure is present on or outside the cells of the fixed animal tissue, e.g., on the surface of a cell of the fixed animal tissue or in the extracellular matrix of the fixed animal tissue.

The term “therapeutic antibody” as used herein refers to any therapeutic antibodies and therapeutic antibody fragments as known in the art. Further, the term is not limited to the therapeutic antibodies and therapeutic antibody fragments as such but also includes conjugates such as antibody drug conjugates.

As referred to herein, the term “small molecule” has the meaning known in the art. Typically, a small molecule to be used in accordance with the invention has a molecular weight of <900 daltons.

In accordance with the present invention, each occurrence of the term “comprising” may optionally be substituted with the term “consisting of”.

The present invention is illustrated by the following non-limiting Examples.

### EXAMPLES

Unless stated otherwise, the following methods were used in the Examples

### Animals Involved in the Study

We used the following mixed-gender animals for the wildDISCO study: 4-week-old wildtype mice (C57BL/6J, CD1 and Balb/c) purchased from Charles River Laboratories. Animals were housed on a 12/12 hr light/dark cycle and had random access to food and water. Temperature was maintained at 18-23° C. and humidity was at 40-60%. Age- and sex-matched C57BL/6J germ-free mice were purchased from the Technical University of Munich (Institute of Nutrition and Health, Core Facility Gnotobiology), and were housed in a germ-free isolator house. The absence of bacteria was confirmed in the germ-free mice by microbial cultures, and mice were then used for further experiments. Each antibody was repeated successfully on at least five mice and also by at least three different person. Animal experiments were performed according to the institutional guidelines of the Ludwig Maximilian University of Munich and the Helmholtz Munich Center German Mouse Clinic after approval of the Ethical Review Board of the Government of Upper Bavaria (Regierung von Oberbayern, Munich, Germany).

### Screening of Cyclodextrin-Containing Buffer for Cholesterol Extraction

Cholesterol extraction was measured using the cholesterol/cholesterol Ester-Glo™ assay (Promega, Madison, USA). 25 mg of PFA fixed mouse liver was incubated in 3 ml of 1% w/v different cyclodextrin-containing antibody buffers: 2-Hydroxypropyl-β-Cyclodextrin (Sigma-Aldrich, H107-100G, LOT WXBC6699V), Methyl-β-cyclodextrin (Sigma-Aldrich, 332615-25G, LOT STBK8343), (2-Hydroxyethyl)-β-cyclodextrin (Sigma-Aldrich, 389137-10G, LOT MKBZ6644V), Triacetyl-β-cyclodextrin (Sigma-Aldrich, 332623-10G, LOT STBJ9765), Succinyl-β-cyclodextrin (Sigma-Aldrich, 85990-500 MG, LOT BCCB7898) and Heptakis (2,6-di-O-methyl)-β-cyclodextrin (Sigma-Aldrich, 39915-1G, LOT BCCF6041), γ-cyclodextrin (Sigma-Aldrich, C4892-5G, LOT SLBJ8855V), α-cyclodextrin (Sigma-Aldrich, 779008-100G, LOT BCCJ0084). The assays were measured at different time points (2 d, 3 d, 5 d, and 7 d). 5 ul aliquot of the supernatant was diluted 10-fold in cholesterol lysis solution and incubated for 30 min at 37° C. Cholesterol detection reagent was then added to the samples and incubated for 60 minutes at room temperature. The value was measured using a Centro LB 96 plate reading luminometer (Berthold, Bad Wildbad, Germany).

### Evaluation of Different Cyclodextrin-Containing Buffer Effect on the Stabilization of Antibody

The homogeneity of antibody, in other words of antibody aggregations, in different cyclodextrin-containing buffers was measured by dynamic light scattering (DLS). TH primary antibody was selected to evaluate antibody stabilization and homogeneity. TH antibody (Millipore, AB152) (Mw: 150 kDa, Concentration: 10 g/l) was dissolved in buffer with and without Heptakis (2,6-di-O-methyl)-β-cyclodextrin (Sigma-Aldrich, 39915-1G) (1% w/v) at room temperature. After 7 days of incubation, the buffer solutions were diluted and afterwards measured in a folded capillary cell (DTS 1070) using a Zetasizer Nano ZS (Malvern, Worcestershire, UK). Samples were measured three times with six sub runs each. The temperature was set to 25° C.

### Permeabilization Capacity of Different Cyclodextrin-Containing Buffer on Half Brain Using Methylene Blue

Mouse half brains were incubated with various cyclodextrin buffers at 1% (w/v), 45 mL for 3 days at 37° C. After PBS wash twice, the samples were added 45 mL of 0.03% methylene blue and incubated overnight at 37° C. To determine the efficiency of methylene blue staining after incubation with different CD buffers, samples were cut in half in the middle line to evaluate the efficacy of the inner tissue staining. The camera images of the samples were analyzed by ImageJ for profile plot along and the pixels were quantified under threshold gray value.

### Perfusion and Whole Mouse Body Fixation

Mice were deeply anesthetized with (0.05 mg/kg fentanyl, 0.5 mg/kg medetomidine, and 5 mg/kg midazolam with intraperitoneal injection) and perfused intracardially with heparinized 0.01 M PBS (10-25 U/ml final heparin concentration, Ratiopharm, N68542.03; perfusion volume 12 ml/minute with an ISMATEC peristaltic pump system). After washing out the blood of the mice for 5-10 minutes, 4% paraformaldehyde (PFA) in 0.01 M PBS (Morphisto, 11762.01000) was perfused 10-20 minutes. The mouse bodies were skinned and transferred to 0.01 M PBS after post fixation in 4% PFA for 6 hours at 4° C.

wildDISCO Whole-Body Immunostaining, PI Labeling and Tissue Clearing

The wildDISCO whole-body immunostaining protocol is mainly based on a setup for pumping the pretreatment solutions and immunostaining buffers through the mouse heart and vasculature to perfuse the whole body. The pumping setup has been previously described^{5,15}. In brief, after post fixation of PFA and 0.1 M PBS washing twice for 30 mins, the mouse body was placed in a 300 ml glass chamber and the perfusion needle was inserted into the mouse heart through the same hole as in PFA perfusion. Then, the perfusion needle was connected to an ISMATEC peristaltic pump (REGLO Digital MS-4/8 ISM 834; reference tube, SC0266), which maintained pressure at 160-230 mmHg (45-60 rpm) and was used to establish transcardiac circulation. The pump was equipped with two channels. One was used to pump the solution through the heart to circulate throughout the mouse, while the second channel collected and circulated the solution leaving the mouse body. In the first channel, a 1-ml syringe tip (Braun, 9166017V) was used to connect the perfusion needle (Leica, 39471024) and the reference tube (Ismatec Reglo, SC0266) which is from the pump and set for circulation of the solution through the heart into the vasculature. Since the second channel allowed the solutions to recirculate, the inflow tubing was immersed in the solution chamber of the glass chamber. After the pump and channels are set up, the needle tip was fixed with superglue (Pattex, PSK1C) to ensure continuing and stable perfusion. All of the following perfusion steps were performed using the setup explained above.

The mice were first perfused with 0.1 M PBS overnight at room temperature, followed by a 2 day perfusion with the decalcification solution containing 10 w/v % EDTA (Carl Roth, 1702922685) in 0.1 M PBS, and the pH was adjusted to 8-9 with sodium hydroxide (Sigma-Aldrich, 71687) to decalcify all bones at room temperature. Then, the mouse body were perfused three times with 0.1 M PBS and washed for 3 hours each time. Next, the mouse was perfused for 1 day with the permeabilization and blocking solution containing 10% goat serum and 2% Triton X-100 in 0.1 M PBS. Then the mice body were perfused with the primary antibodies TH (Millipore, AB152), PGP9.5 (Proteintech, 14730-1-AP), LYVE1 (Thermo Fisher Scientific, 14-0443-82), CD45 (BD Biosciences, 550539), PROX1 (Abcam, ab101851), Podoplanin (Abcam, ab109059) (25 μg in 250 ml, diluted 1:10,000) or 290 μl PI (stock concentration 1 mg ml-1) incubated for 7 days with the 250 ml immunostaining buffer containing 3% goat serum, 10% CHAPS, 2% Triton X-100, 10% DMSO, 1% glycine, 1% Heptakis (2,6-di-O-methyl)-beta-cyclodextrin in 0.1 M PBS. The mouse body was then washed three times in 0.1 M PBS and each time proceeded 12 hours at room temperature. Then, the mice bodies were perfused in the immunostaining buffer at room temperature with the Alexa fluorescent dye-conjugated secondary antibodies: Alexa Fluor 647 goat anti-rabbit IgG antibody (Thermo Fisher Scientific, A-21245) or Alexa Fluor 647 goat anti-rat IgG antibody (Thermo Fisher Scientific, A-21247) (25 μg in 250 ml, diluted 1:10,000) for 7 days. The mice bodies were washed three times with 0.1 M PBS, each time 12 hours.

After the immunostaining steps are done, the mice were transferred to a fume hood and were cleared using the 3DISCO passive whole-body clearing protocol as previous reported9. Briefly, mice bodies were placed in a 300 ml glass chamber and immersed in 200 ml of the following gradient of THF (Tetrahydrofuran, Roth, CP82.1) in distilled water with gentle shaking: (50%×1, 70%×1, 80%×1, 100%×2, 12 h for each step), followed by 3 h in dichloromethane (DCM, Sigma, 270997) and finally in BABB solution (benzyl alcohol+benzyl benzoate 1:2, Sigma, 24122 and W213802) until the bodies were optical transparent.

### Light-Sheet Microscopy Imaging

Image stacks were acquired using a Blaze ultramicroscope (LaVision BioTec GmbH, version 7.3.2) with an axial resolution of 4 μm and the following filter sets: ex 470/40 nm, em 535/50 nm; ex 545/25 nm, em 605/70 nm; ex 640/40 nm, em 690/50 nm. Whole mouse bodies were scanned individually with an Ultramicroscope Blaze light sheet microscopy 4× objective (Olympus XLFLUOR 4× corrected/0.28 NA [WD=10 mm]). We covered the entire mouse in with 9×23 tile scans with 20% overlap and imaged separated from the ventral and dorsal surfaces to a depth of 10 mm, covering the entire body volume with a Z-step of 10 μm. The width of the light-sheet was reduced to 60% to achieve maximum illumination of the field of view, and the exposure time was set to 120 ms. The laser power was adjusted as a function of the intensity of the fluorescence signal to avoid saturation. The acquired raw images TIFF were processed with the Fiji stitching plugin (http://www.discotechnologies.org/).

### Virtual Reality (VR) Headset Operation

Movies require a virtual reality (VR) headset. To visualize them, a VR-Movie player on a VR-device or computer is needed. Movies played in virtual reality need to have “_360” at the end of their file name and set to a “360°/3D” view in the VR-player for an immersive experience.

### Reconstructions of Full-Body Scans and Quantification

Detailed step-by-step instructions for image data stitching and volume fusion were provided previously¹⁵. Briefly, image stacks were recorded using ImSpector software (LaVision BioTec GmbH) and saved in TIFF format for each channel separately. The scanned ventral and dorsal-mouse image data were first stitched using the Fiji stitching plugin and volumes fused using Vision4D (v3.5 x64, Arivis AG, version 3.4.0). To increase the precision of volume fusion, alignment was performed by manually selecting 3 to 4 anatomical landmarks from the overlapping regions. Representative images were created using Imaris (Bitplane AG, version 9.6.0) and Vision4D for 3D volumetric reconstruction, maximum intensity projection, and depth color rendering. To isolate a specific tissue region, the Imaris surface tool was used manually and the mask channel option for pseudocolor was selected. After manual segmentation, the region was visualized in 2D slices using the Ortho Slicer tool. Virtual reality (VR) pictures and movies were generated using the Syglass software (IstoVisio, Inc, version 1.7.2). For quantification of the myenteric plexus in the duodenum between germ free mice and wild type mice, five 200 μm×200 μm×200 μm cubic volumes along the portal triads were randomly selected from the reconstructed 3D images in Imaris. The length of the PGP 9.5-positive myenteric plexus in each cubic volume was traced using Imaris Filament Tracer.

### Quantification

Data are presented as mean±s.d. Statistical analysis was performed using Prism GraphPad software v.8 with 95% confidence interval. P values were calculated using two-tailed unpaired t test to compare data between two groups. P values of <0.05 were considered statistically significant.

### Example 1: Cholesterol Extraction; Penetration of Methylene Blue into the Whole Mouse Brain; Prevention of Antibody Aggregate Formation

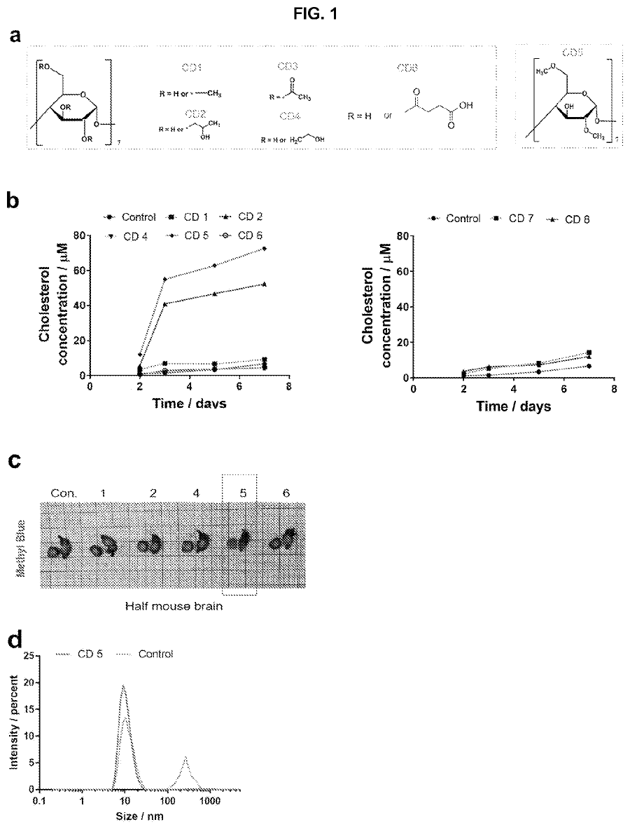

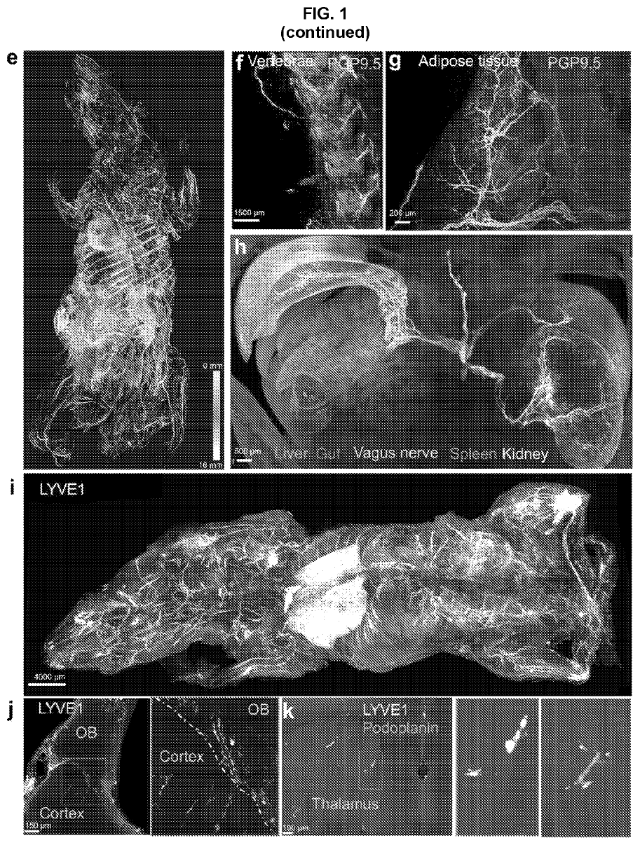

The present inventors investigated the potential ability of β-cyclodextrin variants with diverse nature and number of R-motifs (e.g., methyl-, hydroxypropyl-, hydroxyethyl-, succinyl and acetyl-) (FIG. 1a) to facilitate cholesterol extraction in fixed samples, in combination with the CHAPS and Triton X-100 surfactants to enhance permeabilization (FIG. 3a). Assessing cholesterol extraction using the cholesterol/cholesterol ester-glo assay, the present inventors found that heptakis (2,6-di-O-methyl)-β-cyclodextrin (CD5) extracted most cholesterol from mouse liver tissue after 7 days (FIG. 1b). CD2 (2-Hydroxy-propyl-β-Cyclodextrin) also exhibited good cholesterol extraction ability.

Addition of CD5 to the permeabilization reactions allowed rapid and homogeneous penetration of methylene blue into the whole mouse brain within 12 h (FIG. 1c and FIG. 3b).

As cyclodextrins have been previously reported to stabilize proteins in solution by preventing aggregation¹⁹, the antibody size in antibody solutions was measured using dynamic light scattering (DLS). After 7 days at room temperature without CD5-containing solution, antibodies showed two peaks in the DLS data, one peak at 11.5 nm presumably for the antibody monomer and a peak for larger sizes, which most likely corresponds to different aggregation states. Addition of CD5 prevented the formation of aggregates (FIG. 1d).

### Example 2: Increased Homogeneity and Depth of Antibody Staining in Whole Mouse Bodies

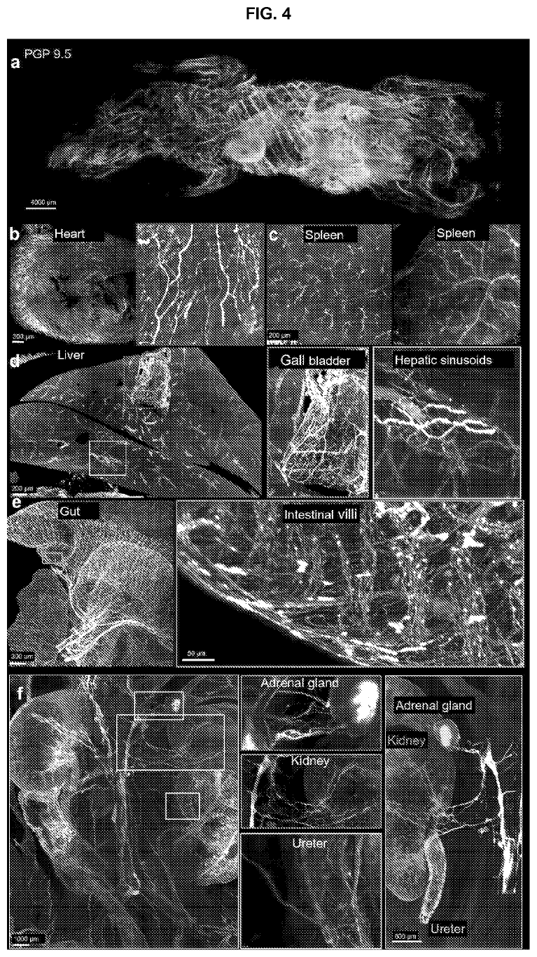

The present inventors next tested if the enhanced membrane permeabilization and the decreased aggregation propensity of antibodies in the CD5-containing buffers increase the homogeneity and depth of antibody staining in whole mouse bodies. Different nerve systems, such as sympathetic and parasympathetic, regulate and coordinate organ function. To reveal delicate nerve innervation of organs in the whole mouse body, the peripheral neuronal network in young adult mouse bodies (˜4 weeks old, ˜10×3×2 cm dimensions) was stained using protein gene product 9.5 (PGP9.5), a pan-neuronal marker (FIG. 1e and FIG. 4a). After whole mouse antibody labeling using CD5-containing buffers, the bodies were rendered optically transparent and performed panoptic imaging using light-sheet microscopy.

The peripheral nerve system was homogeneously stained with no systematic differences in signal intensity between tissues as different as vertebrae (FIG. 1f) and adipose tissue (FIG. 1g) and at different depths of the mouse body.

In the heart, e.g., the network of nerve fibers coursing through the ventricular myocardium was evident (FIG. 4b). The splenic parenchyma showed nerve fibers' complex, panicle-like architecture (FIG. 4c). The vagus nerves branched into smaller fiber bundles as it progressed towards the dorsal spleen, where we also visualized the splenic neural network. PGP9.5+ nerve fibers also innervated the hepatic sinusoids (FIG. 4d) and distributed along the hepatic duct end-to-end. In the gallbladder, the ganglionated plexus comprising a series of irregularly shaped ganglia (FIG. 4d) are clearly visible. In the small intestine, the interconnected ganglionated plexuses on the intestinal wall was observed (FIG. 4e).

In 3D-reconstruction of scans, it was possible to readily observe nerves innervating diverse organs. For example, it was possible to trace vagus nerves, which provides parasympathetic innervation to the abdominal organs, that connects visceral organs, such as the kidneys, adrenal gland, ureter, liver, spleen, and gastrointestinal (GI) tract (FIG. 1h and FIG. 4f), a task that was greatly facilitated with virtual reality visualization techniques. Compared with the whole-organ antibody staining, whole mouse body tracing enabled to visualize nerve connections between different organs (FIG. 1h). This will provide essential clues for understanding the role of nerve communication in normal physiology and disease.

### Example 3: Staining of Lymphatic Vessels and Immune Cells

To show the generalizability of the approach, lymphatic vessels and immune cells were stained using the lymphatic vessel endothelial hyaluronan receptor 1 (LYVE-1) and CD45, respectively.

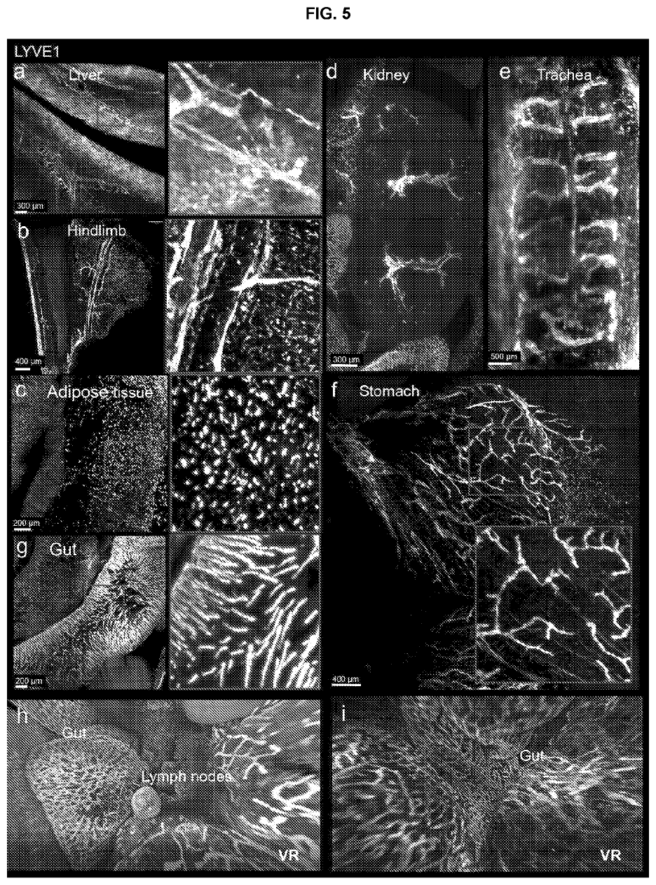

At the whole-mouse level, the finely structured lymphatic network was observed throughout every part of the body (FIG. 1i). It was possible to visualize details of lymph vessel organization in individual organs. For example, LYVE-1+ vessels were observed in the hepatic sinusoidal endothelium and the superficial gastrocnemius (FIG. 5a and FIG. 5b). LYVE-1+ lymph nodes could be observed near the hindlimbs. Especially in adipose tissue, differentially shaped LYVE-1+ cells are seen (FIG. 5c). The larger lymphatic vessels of the kidney branched into lymphatic capillaries with a tree-like architecture (FIG. 5d). Tracheal lymphatic vessels showed a segmental pattern of interconnected vessels (FIG. 5e). In the stomach, lymphatics were unevenly distributed on the gastric walls and had tree-like branches (FIG. 5f). Blunt-ended, tube-like lymphatic capillaries (lacteals) 20 were clearly located in the intestinal villi, and the abundant and well-organized lymphatic plexuses and networks were visible on the outer surface of the intestinal wall (FIG. 5g-5i).

Previously, the brain parenchyma has been proposed to be devoid of lymphatic vessels^{21, 22}, although there is lymphatic drainage from the CNS via meningeal lymphatic vessels. Our whole-body immunolabeling data showed small and short lymphatic capillaries entering the brain parenchyma from the meninges. Some LYVE-1+ lymphatic vessels are also observed to connect the olfactory bulb and the cortex (FIG. 1j), which was observed by LYVE1 and PROX1 staining, respectively. We also find lymph vessels entering the brain parenchyma around thalamus (FIG. 1k), which was confirmed by both with LYVE1 and Podoplanin staining.

### Example 4: Studying the Relationship of Different Physiological Systems in the Same Mouse

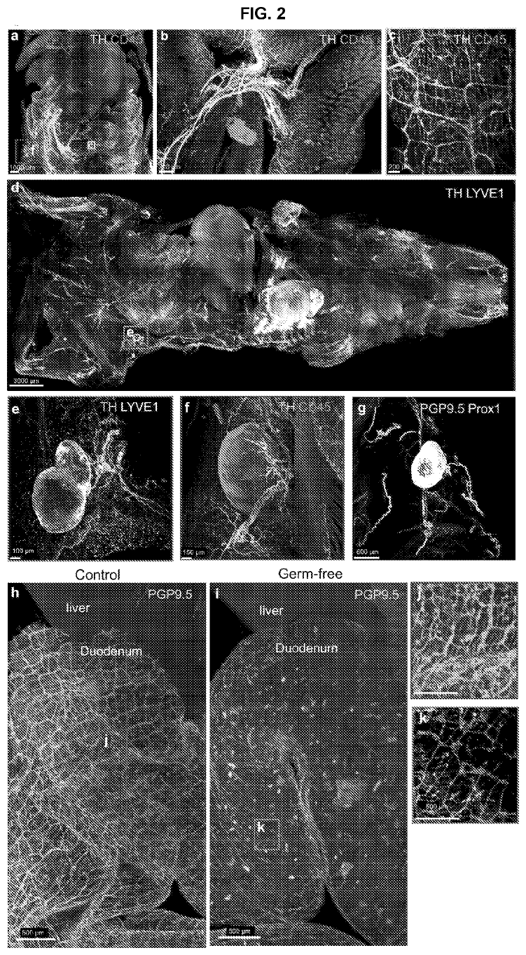

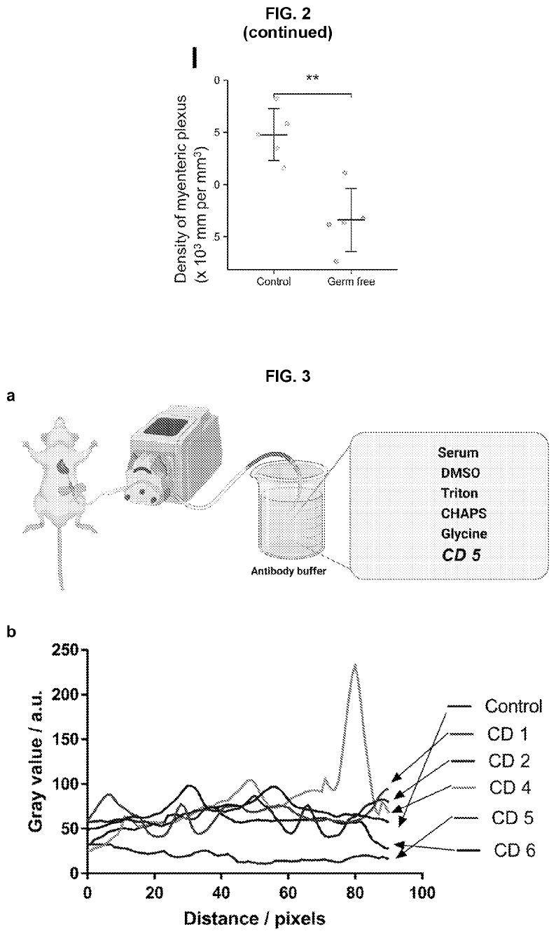

The advantage of wildDISCO compatibility with conventional validated antibodies for labeling allowed studying the relationship of different physiological systems in the same mouse. First, tyrosine hydroxylase (TH)+ sympathetic nerves and CD45+ immune cells were co-immunolabeled (FIG. 2a-2c, FIG. 6). Substantial co-localization of immune cells was found along parts of the vagus nerve, especially at the inferior mesenteric plexus (FIG. 2a and FIG. 2b), and frequent contacts between immune cells and sympathetic nerves on the intestinal wall (FIG. 2c).

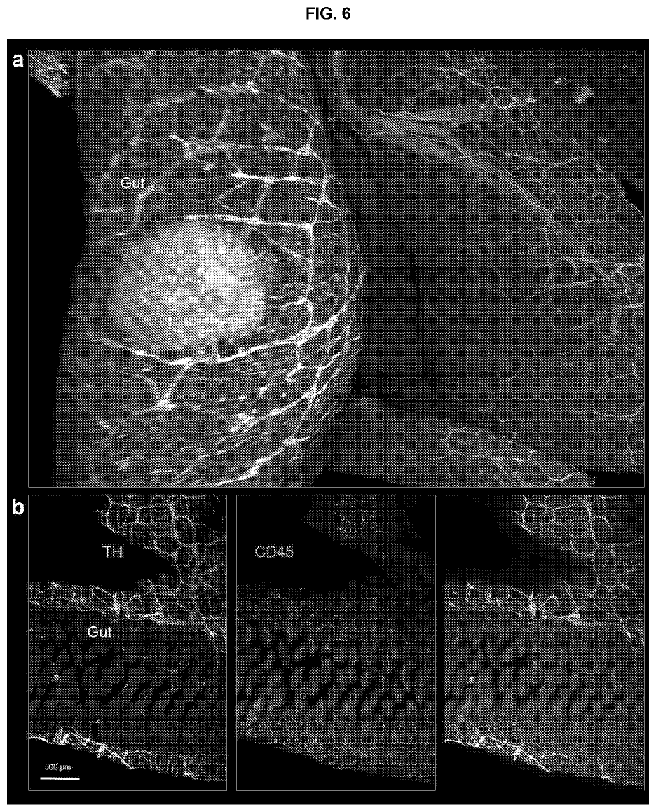

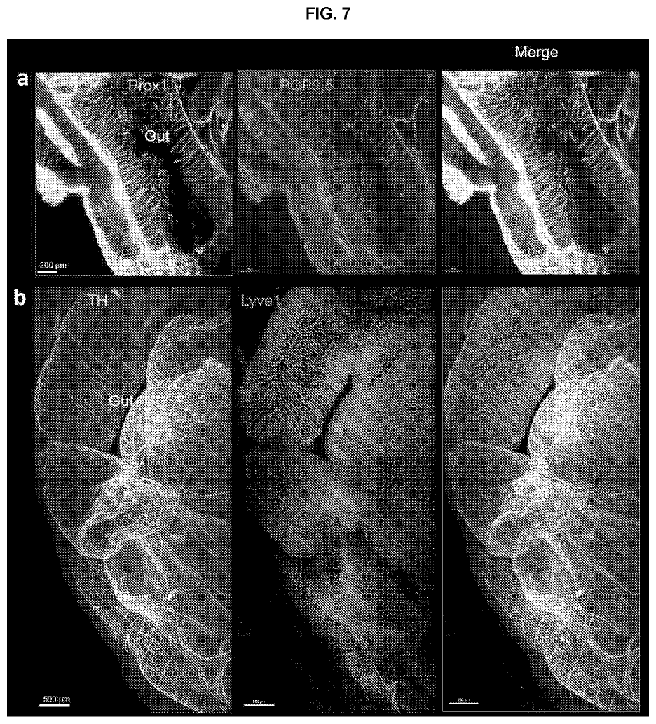

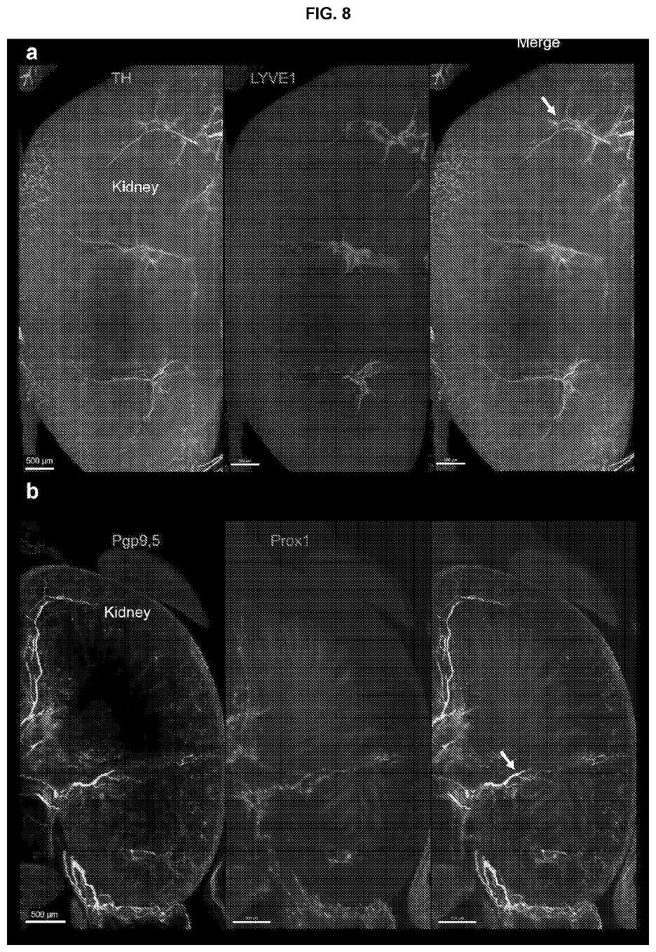

To better illustrate neuro-immune interactions in the lymphatic system, especially the lymph nodes (LN), double-staining of nerve fibers and lymphatic vessels was employed (FIG. 2d, FIG. 7-8). Among others, large LYVE1+ (FIG. 2e) and PROX1+ (Prospero homeobox protein 1, a marker for lymphatic endothelium) (FIG. 2g) LN were detected in the mouse hindlimbs (FIG. 2e, FIG. 2g). CD45 staining confirmed that the observed structures are LNs (FIG. 2f). Co-staining with the pan-neuronal marker PGP9.5 or the peripheral sympathetic neuronal marker TH showed neuronal processes innervating the LN.

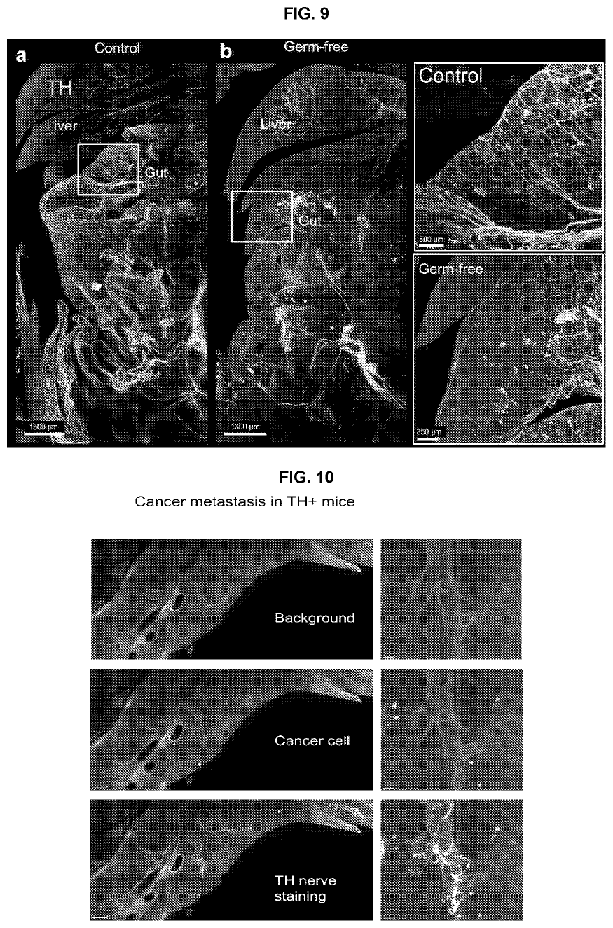

Next, wildDISCO was used to assess the effects of biological perturbations. To this end, the structure of the gut-associated nerve system of germ-free mice was compared to specific-pathogen-free (SPF) standard mice. The double staining data of nerve with lymphatics and nerve with immune cells already showed the enteric nervous system gut intricate details throughout the gut (FIG. 2a-2d). Studying germ-free mice, the PGP9.5+ nerve lattice network was found to be substantially less dense compared to wildtype mice. The density of myenteric plexus reduced from 1.478 (×10³ mm per mm³) to 0.659 (×10³ mm per mm³) (FIG. 2h-21, FIG. 9) confirming the importance of gut microbiota interaction for development and/or maintenance of the mesenteric plexus²³.

### Example 5: WildDISCO Applied into Cancer Metastasis Model

Tyrosine hydroxylase TH was co-stained with breast cancer cells MDA-MB-231. The cancer cells were clearly visible closely around the TH+ nerves. Results are shown in FIG. 10.

### Example 6: WildDISCO Reveals Whole Mouse Artery Networks

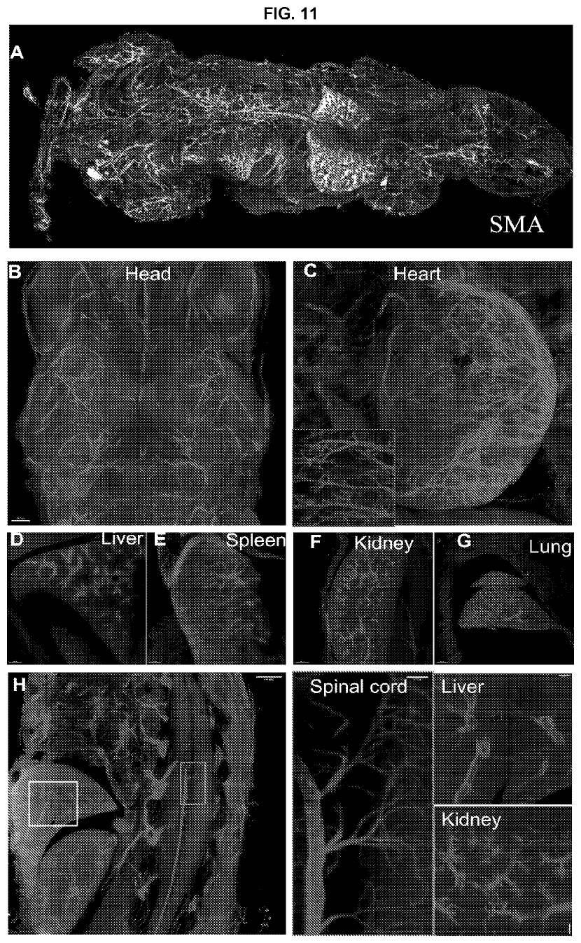

Alpha Smooth Muscle Actin (alpha-SMA) was applied to investigate the distribution of arteries at the whole-body scale. Results are shown in FIG. 11.

### Example 7: Validation of Further Antibodies

To show the broad applicability of the approach, homogeneity and depth of antibody staining was confirmed with further antibodies using the methods described above. A list of validated antibodies is shown in Table 2 below.

In summary, a new DISCO technique (“wildDISCO”) is provided, which allows high-resolution 3D imaging of the peripheral nervous system (PNS), lymphatic system, and vascular system in the whole mouse body.

Most diseases involve multiple interconnected physiological systems, but histological evaluation of their pathology is currently limited to small tissue samples. Herein disclosed is wildDISCO, a technology that involves cholesterol extraction to enable deep tissue penetration of large fluorochrome-containing labeling agents, e.g., standard 150 kDa IgG antibodies, in chemically fixed whole mice. By combining wildDISCO with whole mouse clearing, whole-body maps of the nervous, immune, and lymphatic systems and show their close interactions throughout the mouse body were generated.

wildDISCO can reveal, for instance, integrated neuronal, vascular, and lymphatic networks. By using the technique, it was possible to observe the PNS innervation in most organs, including the heart, lung, liver, kidney, stomach, and intestine. Moreover, the vagal nerves innervate the gastrointestinal tract was visualized. By using the technique, also the lymphatic capillaries heterogeneously penetrated in the center of intestine villi and regional specific 3D villi lymphatics network were presented. Surprisingly, it was found that lymph nodes were innervated by a population of PN with immunomodulatory potential. By using the technique, also the organ-specific vascular patterns and a network of transcortical capillaries as the main support for multiple bones were imaged.

Thus, mapping whole mouse body systems can provide a roadmap for diverse studies, including the neural circuits, immunomodulation, and angiogenesis in the entire mammalian body.

The present invention allows unbiased imaging of transparent whole mouse bodies at cellular resolution provides a comprehensive view of biological systems (nerve or lymphatic systems) in health and disease. WildDISCO does not rely on the transgenic expression of fluorescent proteins as it permits the use of off-the-shelf IgG antibodies to homogenously and simultaneously staining structures in the whole mouse body. The mouse head is a perfect example of the versatility of the method as it combines hard (skull) and soft tissues (brain). Using wildDISCO, it is possible to map all the lymphatic vessels in and around the brain parenchyma in intact mouse heads.

The wildDISCO technology achieves a homogeneous and simultaneous antibody staining throughout the entire mouse bodies. Previously inaccessible 3D anatomical information becomes possible (e.g., aided by the VR visualization), allowing a more comprehensive understanding of the initiation, progression, and extent of pathologies at the whole organism level in mice.

### INDUSTRIAL APPLICABILITY

The methods and products of the present invention are industrially applicable and can, for instance, be used to test biopharmaceutical drugs such as therapeutic antibodies.

### REFERENCES

1. Richardson, D. S. et al. Tissue clearing. Nature Reviews Methods Primers 1, 84 (2021).
2. Ueda, H. R. et al. Tissue clearing and its applications in neuroscience. Nature Reviews Neuroscience 21, 61-79 (2020).
3. Chung, K. et al. Structural and molecular interrogation of intact biological systems. Nature 497, 332-337 (2013).
4. Livet, J. et al. Transgenic strategies for combinatorial expression of fluorescent proteins in the nervous system. Nature 450, 56-62 (2007).
5. Cai, R. et al. Panoptic imaging of transparent mice reveals whole-body neuronal projections and skull-meninges connections. Nature Neuroscience 22, 317-327 (2019).
6. Rios, A. C. et al. Intraclonal Plasticity in Mammary Tumors Revealed through Large-Scale Single-Cell Resolution 3D Imaging. Cancer Cell 35, 618-632.e616 (2019).
7. Yang, B. et al. Single-Cell Phenotyping within Transparent Intact Tissue through Whole-Body Clearing. Cell 158, 945-958 (2014).
8. Park, Y.-G. et al. Protection of tissue physicochemical properties using polyfunctional crosslinkers. Nature Biotechnology 37, 73-83 (2019).
9. Ku, T. et al. Elasticizing tissues for reversible shape transformation and accelerated molecular labeling. Nature Methods 17, 609-613 (2020).
10. Murray, E. et al. Simple, Scalable Proteomic Imaging for High-Dimensional Profiling of Intact Systems. Cell 163, 1500-1514 (2015).
11. Renier, N. et al. iDISCO: A Simple, Rapid Method to Immunolabel Large Tissue Samples for Volume Imaging. Cell 159, 896-910 (2014).
12. Ertürk, A. et al. Three-dimensional imaging of solvent-cleared organs using 3DISCO. Nature Protocols 7, 1983-1995 (2012).
13. Susaki, Etsuo A. et al. Whole-Brain Imaging with Single-Cell Resolution Using Chemical Cocktails and Computational Analysis. Cell 157, 726-739 (2014).
14. Dodt, H.-U. et al. Ultramicroscopy: three-dimensional visualization of neuronal networks in the whole mouse brain. Nature Methods 4, 331-336 (2007).
15. Zhao, S. et al. Cellular and Molecular Probing of Intact Human Organs. Cell 180, 796-812.e719 (2020).
16. Belle, M. et al. Tridimensional Visualization and Analysis of Early Human Development. Cell 169, 161-173.e112 (2017).
17. Feng, G. et al. Imaging Neuronal Subsets in Transgenic Mice Expressing Multiple Spectral Variants of GFP. Neuron 28, 41-51 (2000).
18. Mahammad, S. & Parmryd, I. in Methods in Membrane Lipids. (ed. D. M. Owen) 91-102 (Springer New York, New York, NY; 2015).
19. Serno, T., Geidobler, R. & Winter, G. Protein stabilization by cyclodextrins in the liquid and dried state. Advanced Drug Delivery Reviews 63, 1086-1106 (2011).
20. Bernier-Latmani, J. & Petrova, T. V. High-resolution 3D analysis of mouse small-intestinal stroma. Nature Protocols 11, 1617-1629 (2016).
21. Cugurra, A. et al. Skull and vertebral bone marrow are myeloid cell reservoirs for the meninges and CNS parenchyma. Science 373, eabf7844 (2021).
22. Louveau, A. et al. Structural and functional features of central nervous system lymphatic vessels. Nature 523, 337-341 (2015).
23. Fülling, C., Dinan, T. G. & Cryan, J. F. Gut Microbe to Brain Signaling: What Happens in Vagus. Neuron 101, 998-1002 (2019).

### FURTHER REFERENCES

Battke, C., Kremmer, E., Mysliwietz, J., Gondi, G., Dumitru, C., Brandau, S., Lang, S., Vullo, D., Supuran, C., and Zeidler, R. (2011). Generation and characterization of the first inhibitory antibody targeting tumour-associated carbonic anhydrase XII. Cancer Immunol Immunother 60, 649-658.
Butler, J. M., Kobayashi, H., and Rafii, S. (2010). Instructive role of the vascular niche in promoting tumour growth and tissue repair by angiocrine factors. Nat Rev Cancer 10, 138-146.
Calvo, C. F., Fontaine, R. H., Soueid, J., Tammela, T., Makinen, T., Alfaro-Cervello, C., Bonnaud, F., Miguez, A., Benhaim, L., Xu, Y., et al. (2011). Vascular endothelial growth factor receptor 3 directly regulates murine neurogenesis. Genes Dev 25, 831-844.
Condeelis, J., and Weissleder, R. (2010). In vivo imaging in cancer. Cold Spring Harb Perspect Biol 2, a003848.
Gage, G. J., Kipke, D. R., and Shain, W. (2012). Whole animal perfusion fixation for rodents. J Vis Exp. Ghanavati, S., Yu, L. X., Lerch, J. P., and Sled, J. G. (2014). A perfusion procedure for imaging of the mouse cerebral vasculature by X-ray micro-CT. J Neurosci Methods 221, 70-77.
Gondi, G., Mysliwietz, J., Hulikova, A., Jen, J. P., Swietach, P., Kremmer, E., and Zeidler, R. (2013). Antitumor efficacy of a monoclonal antibody that inhibits the activity of cancer-associated carbonic anhydrase XII. Cancer Res 73, 6494-6503.
Holliger, P., and Hudson, P. J. (2005). Engineered antibody fragments and the rise of single domains. Nat Biotechnol 23, 1126-1136.
Hong, G., Antaris, A. L., and Dai, H. (2017). Near-infrared fluorophores for biomedical imaging. Nature Biomedical Engineering 1, 0010.
Iorns, E., Drews-Elger, K., Ward, T. M., Dean, S., Clarke, J., Berry, D., El Ashry, D., and Lippman, M. (2012). A new mouse model for the study of human breast cancer metastasis. PloS one 7, e47995.
Janeway, C. A., Travers, P., Walport, M., and Shlomchik, M. J. (1997). Immunobiology: the immune system in health and disease, Vol 1 (Current Biology).
Louveau, A., Smirnov, I., Keyes, T. J., Eccles, J. D., Rouhani, S. J., Peske, J. D., Derecki, N. C., Castle, D., Mandell, J. W., Lee, K. S., et al. (2015). Structural and functional features of central nervous system lymphatic vessels. Nature 523, 337-341.
Massoud, T. F., and Gambhir, S. S. (2003). Molecular imaging in living subjects: seeing fundamental biological processes in a new light. Genes Dev 17, 545-580.
Massoud, T. F., and Gambhir, S. S. (2007). Integrating noninvasive molecular imaging into molecular medicine: an evolving paradigm. Trends Mol Med 13, 183-191.
Muyldermans, S. (2013). Nanobodies: natural single-domain antibodies. Annu Rev Biochem 82, 775-797. Ntziachristos, V. (2010). Going deeper than microscopy: the optical imaging frontier in biology. Nat Methods 7, 603-614.
Pan, C., Cai, R., Quacquarelli, F. P., Ghasemigharagoz, A., Lourbopoulos, A., Matryba, P., Plesnila, N., Dichgans, M., Hellal, F., and Erturk, A. (2016). Shrinkage-mediated imaging of entire organs and organisms using uDISCO. Nat Methods.
Pandey, M., and Mahadevan, D. (2014). Monoclonal antibodies as therapeutics in human malignancies. Future Oncol 10, 609-636.
Chapter 7 of Paul, W. E. (Ed.).: Fundamental Immunology 2nd Ed. Raven Press, Ltd., New York 1989 Pichler, B. J., Wehrl, H. F., and Judenhofer, M. S. (2008). Latest advances in molecular imaging instrumentation. J Nucl Med 49 Suppl 2, 5S-23S.
Ransohoff, R. M., and Engelhardt, B. (2012). The anatomical and cellular basis of immune surveillance in the central nervous system. Nat Rev Immunol 12, 623-635.
Timpson, P., McGhee, E. J., and Anderson, K. I. (2011). Imaging molecular dynamics in vivo—from cell biology to animal models. J Cell Sci 124, 2877-2890.
Tuchin, V. V. (2016). Editor's Introduction: Optical Methods for Biomedical Diagnosis.
Vick, B., Rothenberg, M., Sandhofer, N., Carlet, M., Finkenzeller, C., Krupka, C., Grunert, M., Trumpp, A., Corbacioglu, S., Ebinger, M., et al. (2015). An advanced preclinical mouse model for acute myeloid leukemia using patients' cells of various genetic subgroups and in vivo bioluminescence imaging. PloS one 10, e0120925.
Welti, J., Loges, S., Dimmeler, S., and Carmeliet, P. (2013). Recent molecular discoveries in angiogenesis and antiangiogenic therapies in cancer. J Clin Invest 123, 3190-3200.
Wilson, E. H., Weninger, W., and Hunter, C. A. (2010). Trafficking of immune cells in the central nervous system. J Clin Invest 120, 1368-1379.
Yoneda, T., Wiliams, P. J., Hiraga, T., Niewolna, M. & Nishimura, R. A bone-seeking clone exhibits different biological properties from the MDA-MB-231 parental human breast cancer cells and a brain-seeking clone in vivo and in vitro. J Bone Miner Res 16, 1486-1495, doi: 10.1359/jbmr.2001.16.8.1486 (2001).
Zipfel, W. R., Williams, R. M., Christie, R., Nikitin, A. Y., Hyman, B. T., and Webb, W. W. (2003). Live tissue intrinsic emission microscopy using multiphoton-excited native fluorescence and second harmonic generation. Proc Natl Acad Sci USA 100, 7075-7080.

1. A method for preparing an animal tissue for fluorescence microscopy, the method comprising:
labeling a target molecule in the fixed animal tissue with a labeling solution comprising a fluorochrome-containing labeling agent capable of binding to said target molecule, said labeling agent having a molecular weight of more than 100 kDa, to obtain a fixed animal tissue labeled with said fluorochrome-containing labeling agent,
wherein the fixed animal tissue is treated with a permeabilization solution, and
wherein the permeabilization solution and/or the labeling solution comprises a cyclodextrin derivative.

2. The method according to claim 1, wherein the cyclodextrin derivative has a structure represented by the following formula: 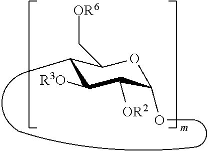
wherein:
m is 6 to 8;
R², R³ and R⁶ are each independently selected from H and optionally substituted alkyl; and
the degree of substitution (DS), representing the average number of non-hydrogen groups R², R³ and R⁶ per glucopyranose unit, is 0 to 3.

3. The method according to claim 2, wherein:
the optionally substituted alkyl is a linear or branched C₁-C₆ alkyl optionally substituted by one or more groups selected from OH, SO₃H, SO₃Na, oxo and COOH; and/or
DS≥0.9; and/or
m is 7.

4. The method according to any one of claims 2 and 3, with the proviso that when R², R³ and R⁶ are each selected from H and CH₃, then DS≥2.

5. The method according to any one of claims 2 to 4, wherein:
R² are R⁶ are CH₃, and R³ is H; or
R², R³ and R⁶ are each independently selected from H and —CH₂CH(OH)CH₃.

6. The method according to claim 1, wherein the cyclodextrin derivative is selected from (2-hydroxypropyl)-β-cyclodextrin, triacetyl-β-cyclodextrin, (2-hydroxyethyl)-β-cyclodextrin, heptakis (2,6-di-O-methyl)-β-cyclodextrin, succinyl-β-cyclodextrin, γ-cyclodextrin and α-cyclodextrin; more preferably (2-hydroxypropyl)-β-cyclodextrin or heptakis (2,6-di-O-methyl)-β-cyclodextrin; most preferably heptakis (2,6-di-O-methyl)-β-cyclodextrin.

7. The method according to any one of the preceding claims, wherein the fluorochrome-containing labeling agent is an antibody conjugated to said fluorochrome, said antibody being capable of binding to said target molecule, preferably wherein the antibody is an IgG, IgA, IgM, IgD or IgE.

8. The method according to any one of the preceding claims, further comprising, prior to labeling the target molecule in the fixed animal tissue with the labeling solution, the following step:
contacting the fixed animal tissue with a primary antibody capable of binding to a structure, preferably a protein, lipid, DNA or RNA, that is present in said fixed animal tissue, more preferably a protein that is present in said fixed animal tissue,
wherein the fluorochrome-containing labeling agent is capable of binding to the primary antibody.

9. The method according to any one of the preceding steps, further comprising:
blocking step for blocking unspecific antigen binding of antibodies, wherein the blocking step is performed by treating the fixed animal tissue with a blocking solution before the step of labeling,
wherein the blocking solution comprises animal serum.

10. The method according to any one of the preceding claims comprising the following steps, in this order:
decolorizing, permeabilizing and blocking step performed by treating the fixed animal tissue with solution(s) for the removal of heme, permeabilization and blocking; and
labeling a target molecule in the fixed animal tissue with a labeling solution comprising the cyclodextrin derivative and the fluorochrome-containing labeling agent capable of binding to said target molecule, said labeling agent having a molecular weight of more than 100 kDa,
to obtain a fixed animal tissue labeled with said fluorochrome-containing labeling agent.

11. Use of a cyclodextrin derivative for improving labeling of a target molecule in a fixed animal tissue or whole animal body with a fluorochrome-containing labeling agent,
wherein the cyclodextrin derivative is defined as in any one of claims 2 to 6,
with the proviso that when R², R³ and R⁶ are each selected from H and CH₃, then DS≥2.

12. The use according to claim 11, wherein the fluorochrome-containing labeling agent has a molecular weight of more than 100 kDa and/or is an antibody conjugated to said fluorochrome, said antibody being capable of binding to said target molecule, preferably wherein the antibody is an IgG, IgA, IgM, IgD or IgE.

13. Composition comprising an antibody having a molecular weight of more than 100 kDa, and a cyclodextrin derivative as defined in any one of claims 2 to 6,
with the proviso that when R², R³ and R⁶ are each selected from H and CH₃, then DS≥2, wherein the antibody is preferably a fluorochrome-containing labeling agent or a primary antibody.

14. The composition according to claim 13, further comprising one or more, preferably all of the following components:
animal serum, preferably mammalian serum, more preferably goat serum;
zwitterionic surfactant, preferably CHAPS or CHAPSO, more preferably CHAPS;
non-ionic surfactant, preferably Triton X-100 or IGEPAL CA-630, more preferably Triton X-100;
organic solvent, preferably a water-miscible solvent, more preferably DMSO; and/or
amino acid, preferably glycine,
in aqueous buffer solution, preferably phosphate buffer saline (PBS).

15. The composition according to claim 14, wherein the components, if present, have the following concentrations:
cyclodextrin derivative: 0.5 to 2, preferably 0.75 to 1.5, more preferably 1% w/v;
animal serum: 0.5 to 12, preferably 0.75 to 10, more preferably 1 to 3% v/v;
zwitterionic surfactant: 5 to 15, preferably 7.5 to 12.5, more preferably 10% w/v;
non-ionic surfactant: 0.5 to 4, preferably 1 to 3, more preferably 2% w/v;
organic solvent: 5 to 20, preferably 7.5 to 15, more preferably 10% w/v;
amino acid: 0.5 to 2, preferably, 0.75 to 1.5, more preferably 1% w/v;
and wherein the aqueous buffer has a buffer agent concentration of 0.05 to 0.2 M, preferably 0.08 to 1.2 M, more preferably 0.1 M.
